(19) United States
(12) Patent Application Publication
(10) Pub. No.: US 2006/0078558 A1
(43) Pub. Date: Apr. 13, 2006

(54) Diagnosis, prognosis and treatment of pulmonary diseases

(76) Inventor: Jeffrey A. Whitsett, Cincinnati, OH (US)

Correspondence Address: Frost Brown Todd LLC, 2200 PNC Center, 201 East Fifth Street, Cincinnati, OH, 45202, US

(21) Appl. No.: 10/772,716

(22) Filed: Feb. 5, 2004

Related U.S. Application Data
(60) Provisional application No. 60/519,453, filed on Nov. 12, 2003.

(51) Int. Cl. A61K 48/00; A61K 39/395

(52) U.S. Cl. 424/143.1; 514/44; 514/2

(57) ABSTRACT

The present invention provides methods to protect a subject from a respiratory disorder involving an airway obstructive disease such as asthma or chronic obstructive pulmonary disease. Provided are methods to protect a subject from an airway obstructive disease using gene therapy. Methods are provided for supplying FoxA2 function to cells of the lung and airway, such as smooth muscle and epithelial cells, by FoxA2 gene therapy. The FoxA2 gene, a modified FoxA2 gene, or a part of the gene may be introduced into the cell in a vector such that the gene remains extrachromosomal or may be integrated into the subjects chromosomal DNA for expression. These methods provide for administering to a subject in need of such treatment a therapeutically effective amount of a FoxA2 gene, or pharmaceutically acceptable composition thereof, for overexpressing the FoxA2 gene. Such methods of expressing the administered FoxA2 gene in the lungs and airway provide for: (1) preventing or alleving bronchial hyperresponsiveness; (2) preventing or alleving of an airway obstructive disease, e.g., bronchial hyperreactivity, airway hyperresponsiveness, asthma or chronic obstructive pulmonary disorder (“COPD”); (3) reducing the airway resistance response to inhaled natural or synthetic bronchoconstrictors or allergens or to exercise; and (4) enhancing responsiveness (relaxation) of airway tissues to β-agonists.

This application claims the benefit of U.S. Provisional Patent Application Ser. No. 60/519,453, filed Nov. 12, 2003, which application is hereby incorporated by reference in its entirety.

This invention was made in part with Government support under Grant No. R01 HL56387, awarded by the National Institutes of Health. The Government may have certain rights in this invention.

### FIELD OF THE INVENTION

The present invention generally relates to a method to protect a mammal from a disease involving inflammation. Accordingly, one object of the present invention is to provide methods for treating and preventing inflammation in vivo, in particular, a respiratory disease involving inflammation.

### BACKGROUND OF THE INVENTION

Diseases involving inflammation are characterized by the influx of certain cell types and mediators, the presence of which can lead to tissue damage and sometimes death. Diseases involving inflammation are particularly harmful when they afflict the respiratory system, resulting in obstructed breathing, hypoxemia, hypercapnia and lung tissue damage. Obstructive diseases of the airways are characterized by airflow limitation (i.e., airflow obstruction or narrowing) due to constriction of airway smooth muscle, edema and hypersecretion of mucous leading to increased work in breathing, dyspnea, hypoxemia and hypercapnia. While the mechanical properties of the lungs during obstructed breathing are shared between different types of obstructive airway disease, the pathophysiology can differ.

A variety of inflammatory agents can provoke airflow limitation including allergens, cold air, exercise, infections and air pollution. In particular, allergens and other agents in allergic or sensitized mammals (i.e., antigens and haptens) cause the release of inflammatory mediators that recruit cells involved in inflammation. Such cells include lymphocytes, eosinophils, mast cells, basophils, neutrophils, macrophages, monocytes, fibroblasts and platelets. Inflammation results in airway hyperresponsiveness. A variety of studies have linked the degree, severity and timing of the inflammatory process with the degree of airway hyperresponsiveness. Thus, a common consequence of inflammation is airflow limitation and/or airway hyperresponsiveness.

Asthma is a significant disease of the lung, which effects nearly 12 million Americans. Asthma is typically characterized by periodic airflow limitation and/or hyperresponsiveness to various stimuli which results in excessive airways narrowing. Other characteristics can include inflammation of airways, eosinophilia and airway fibrosis.

Airway fibrosis due to the deposition of collagen or provisional matrix beneath the basement membrane is a consistent finding in asthma patients, even in the airways of patients with mild asthma. This deposition of collagen is not altered by steroid treatment. Clinical studies have shown a positive correlation between airway fibrosis and airway dysfunction (e.g., airflow limitation or airways hyperresponsiveness). The inflammatory mechanisms which result in this collagen deposition are unknown and more importantly, the functional significance of airway fibrosis is not understood. There is a need to determine the mechanisms which link inflammation, airways remodeling and pathophysiology in asthma since such mechanisms are likely to have a bearing on disease severity and the efficaciousness of therapeutics, as well as their role in other inflammatory diseases.

Currently, therapy for treatment of inflammatory diseases such as moderate to severe asthma predominantly involves the use of glucocorticosteroids. Other anti-inflammatory agents that are used to treat inflammatory diseases include cromolyn and nedocromil. Symptomatic treatment with beta-agonists, anticholinergic agents and methyl xanthines are clinically beneficial for the relief of discomfort but fail to stop the underlying inflammatory processes that cause the disease. The frequently used systemic glucocorticosteroids have numerous side effects, including, but not limited to, weight gain, diabetes, hypertension, osteoporosis, cataracts, atherosclerosis, increased susceptibility to infection, increased lipids and cholesterol, and easy bruising. Aerosolized glucocorticosteroids have fewer side effects but can be less potent and have significant side effects, such as thrush.

Other anti-inflammatory agents, such as cromolyn and nedocromil are much less potent and have fewer side effects than glucocorticosteroids. Anti-inflammatory agents that are primarily used as immunosuppressive agents and anti-cancer agents (i.e., cytoxan, methotrexate and Immuran) have also been used to treat inflammation with mixed results. These agents, however, have serious side effect potential, including, but not limited to, increased susceptibility to infection, liver toxicity, drug-induced lung disease, and bone marrow suppression. Thus, such drugs have found limited clinical use for the treatment of most airway hyperresponsiveness lung diseases.

The use of anti-inflammatory and symptomatic relief reagents is a serious problem because of their side effects or their failure to attack the underlying cause of an inflammatory response. There is a continuing requirement for less harmful and more effective reagents for treating inflammation. Thus, there remains a need for processes using reagents with lower side effect profiles and less toxicity than current anti-inflammatory therapies.

### SUMMARY OF THE INVENTION

The present invention relates to compounds and their uses, particularly in the pharmaceutical industry. The invention discloses compounds having anti-inflammatory activities, as well as methods for treating various diseases associated with inflammation. It further deals with pharmaceutical compositions comprising said compounds, more particularly useful to treat pulmonary disease.

The present invention also provides for enhancement of expression or replacement of FoxA2, whether by gene transfer vectors to express the normal allele or protein replacement with purified FoxA2 or recombinant FoxA2 or FoxA2 analogues or by treatment with agents that increase or maintain levels of FoxA2 in airway epithelial cells, are beneficial for the treatment of pulmonary disorders.

The pathology of the lung disease includes idiopathic pulmonary fibrosis (IPF), desquamating interstitial pneumonitis (DIP), usual interstitial pneumonitis (UIP), non-specific interstitial pneumonitis (NSIP), and other forms of interstitial lung disease.

FoxA2 may be administered by aerosol or inhalation of a pharmaceutically useful preparation containing surfactant-like phospholipids, including phosphatidylglycerol and phosphatidylcholine.

The present invention provides for the use of a diagnostic screening tool based on the absence or abnormalities of FoxA2 in tissues or lavage lung material using immunohistochemistry, ELISA, Western blots, Mass spectroscopy, and protein or gene sequencing and genotyping. High throughput screening of chemical compounds can be used to determine molecular structures that increase or maintain FoxA2 expression levels (RNA and protein). Optimized compounds can then be used systemically or topically to treat lung disease.

The present invention provides methods to protect a subject from a respiratory disorder involving an airway obstructive disease such as asthma or chronic obstructive pulmonary disease.

FoxA1 is expressed in the lung in sites overlapping with that of FoxA2. Deletion of FoxA1 in the mouse results in birth of mice with a relatively normal lung structure, but CCSP expression is lacking in bronchiolar epithelial cells, indicating that FoxA1 regulates normal respiratory epithelial cell differentiation. Deletion of FoxA2 causes abnormalities in gene expression, and results in airway epithelial remodeling postnatally. FoxA1 and FoxA2 share near identity in the DNA binding domain, but diverge considerably (approximately 50-60%) in immuno- and carboxy-terminal activation domains.

Factors or active agents that result in increased FoxA1/FoxA2 expression can be used as a therapeutic strategy to normalize lung epithelial cell differentiation associated with inflammatory or hereditary lung disease. Since FoxA2 controls goblet cell differentiation, its expression or agents, which enhance or stabilize its expression or activity may also control epithelial cell function and differentiation. Furthermore, since FoxA1 and FoxA2 control a number of proteins involved in host defense, antioxidant surfactant homeostasis, and lung repair, active agents or methods to enhance or regulate their activities, are useful in the prevention or therapy of various inflammatory and hereditary lung diseases, including cystic fibrosis, asthma, emphysema, pulmonary fibrosis, bronchiectasis, and recurrent infection.

In one embodiment, drugs such as FK506 (an immunosuppressive agent, USAN tacrolimus; Prograf) are used to activate or regulate FoxA1/FoxA2 expression for therapy of various pulmonary disorders. Alternatively, antisense or gene constructs, which inhibit or stimulate Foxa expression, are used as therapy for long-term disorders such as cystic fibrosis.

In one embodiment, provided are methods to protect a subject from an airway obstructive disease using gene therapy. Methods are provided for supplying FoxA2 function to cells of the lung and airway, such as smooth muscle and epithelial cells, by FoxA2 gene therapy. The FoxA2 gene, a modified FoxA2 gene, or a part of the gene may be introduced into the cell in a vector such that the gene remains extrachromosomal or may be integrated into the subjects chromosomal DNA for expression. These methods provide for administering to a subject in need of such treatment a therapeutically effective amount of a FoxA2 gene, or pharmaceutically acceptable composition thereof, for overexpressing the FoxA2 gene. Such methods of expressing the administered FoxA2 gene in the lungs and airway provide for: (1) preventing or alleving bronchial hyperresponsiveness; (2) preventing or alleving of an airway obstructive disease, e.g., bronchial hyperreactivity, airway hyperresponsiveness, asthma or chronic obstructive pulmonary disorder (“COPD”); (3) reducing the airway resistance response to inhaled natural or synthetic bronchoconstrictors or allergens or to exercise; and (4) enhancing responsiveness (relaxation) of airway tissues to β-agonists.

The FoxA2 gene or a part of the gene may or may not be integrated (covalently linked) to chromosomal DNA making up the genome of the subject's target cells. The genes may be introduced into the cell in a vector such that the gene remains extrachromosomal. In such a situation, the gene will be expressed by the cell from the extrachromosomal location. The cells may also be transformed where the exogenous DNA has become integrated into the chromosome so that it is inherited by daughter cells through chromosome replication. The gene may be introduced into an appropriate vector for extrachromosomal maintenance or for integration into the host. Vectors for introduction of genes or introduction of the FoxA2 protein or peptide (e.g., the protein fusion tat-FoxA2, etc.) both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of those in the art.

The polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the mature polypeptide may be identical to the coding sequence shown in FIG. 1, (SEQ ID NO:1 and SEQ ID NO:3) or may be a different coding sequence which coding sequence, as a result of the redundancy or degeneracy of the genetic code, encodes the same mature polypeptide as the DNA of FIG. 1 (SEQ ID NO:2).

In another embodiment, provided are methods to genotype a subject for FoxA2/FoxA1 alleles for determining the susceptibility to bronchial hyperresponsiveness; an airway obstructive disease, e.g., bronchial hyperreactivity, airway hyperresponsiveness, asthma or chronic obstructive pulmonary disorder (“COPD”), or other lung disease.

The polynucleotide which encodes for the mature FoxA2 polypeptide of FIG. 1 (SEQ ID NO:2) may include: only the coding sequence for the mature polypeptide; the coding sequence for the mature polypeptide and additional coding sequence; the coding sequence for the mature polypeptide (and optionally additional coding sequence) and non-coding sequence, such as introns or non-coding sequence 5′ and/or 3′ of the coding sequence for the mature polypeptide.

### BRIEF DESCRIPTION OF THE DRAWINGS

FIG. 1. A schematic drawing showing the sequence homology between the two human variants of FoxA2 by comparing nucleotide matching of Homo sapiens forkhead box A2 (FoxA2), transcript variant 1, mRNA, nucleotides 3 through 2242 (SEQ ID NO:1; GenBank Accession No. NM_{—}021784.3) with that of Homo sapiens forkhead box A2 (FoxA2), transcript variant 2, mRNA, nucleotides 2 through 2230 (SEQ ID NO:3; GenBank Accession No. NM_{—}153675.1). Variant 2 differs in 5′ UTR compared to variant 1. Variant 1 has 2 exons and variant 2 has 3 exons. Both map to chromosome 20 contig NT_{—}011387.

FIG. 2. A. Conditional control of FoxA2 gene targeting. In triple transgenic mice (FoxA2-rtTA-/tg, (tetO)7CMV-Cre-/tg, FoxA2loxP/loxP and CCSPrtTA-/tg, (tetO)7CMV-Cre-/tg, FoxA2loxP/loxP), rtTA is expressed in epithelial cells under control of human FoxA2 or CCSP promoter. In the presence of doxycycline, rtTA binds to (tetO)7CMV promoter and activates the expression of Cre-recombinase, causing recombination and deletion of exon 3 in FoxA2loxP/loxP mice, producing FoxA2Δ/Δ mice. B. Immunohistochemistry demonstrates FoxA2 deletion. Lung sections are prepared on PN16 for immunohistochemistry using anti-FoxA2 antibody. Triple transgenic mice, FoxA2-rtTA-/tg, (tetO)7CMV-Cre-/tg, FoxA2loxP/loxP (B) or CCSPrtTA-/tg, (tetO)7CMV-Cre-/tg, FoxA2loxP/loxP (D), and littermate control mice (A, C) are maintained on doxycycline from E0. Inserts are higher magnifications (×40) of the indicated regions (arrowhead). Nuclear FoxA2 staining is observed in epithelial cells of conducting and peripheral airways and alveoli, and is absent or decreased in FoxA2Δ/Δ mice. Goblet cell hyperplasia is observed in both FoxA2- and CCSP-rtTA FoxA2Δ/Δ mice (inset). Figures are representative of at least four individual mice (bar=50 μm). C. RNA protection assay for estimation of FoxA2 mRNA. RNA protection assay is used to quantitate FoxA2 mRNA in lungs from FoxA2-rtTA, FoxA2Δ/Δ and control littermates at E18.5 and compared with L32 mRNA. Dams are treated with doxycycline from E0 to E18.5.

FIG. 3. Effects of FoxA2 deletion on lung morphogenesis. Triple transgenic mice (FoxA2-rtTA-/tg, (tetO)7CMV-Cre-/tg, FoxA2loxP/loxP) and littermate control mice are maintained on doxycycline from E0. Lung sections of triple transgenic mice and littermate controls are prepared on E18.5 (A, B), PN3 (C, D) and PN16 (E, F) and stained with hematoxylin-eosin to assess lung morphology. Figures are representative of at least 4 individual mice (bar=300 μm).

FIG. 4. Morphometric analysis. Fractional airspace and fractional lung parenchyma are calculated in lungs of mice from each genotype after exposure to doxycycline for the defined times. Fractional airspace ratio is significantly increased in FoxA2-rtTA but not CCSP-rtTA, Fox2Δ/Δ mice. *P<0.05 vs. control mice (by Student's t-test).

FIG. 5. Goblet cell hyperplasia after FoxA2 deletion. Compound transgenic mice, FoxA2-rtTA-/tg, (tetO)7CMV-Cre-/tg, FoxA2loxP/loxP or CCSPrtTA-/tg, (tetO)7CMV-Cre-/tg, FoxA2loxP/loxP and controls (non-FoxA2 deleted) are treated with doxycycline from E0. At PN16, lung sections are stained for FoxA2 (arrow) and Alcian blue or periodic acid Schiff (PAS). Staining by Alcian blue is observed in cells with goblet cell morphology, arrowhead (A, B, C, D). Similarly, PAS (arrowhead E, F) and mucin5A/C staining (arrowhead G, H) are observed in goblet cells in conducting airways in FoxA2 deleted mice. FoxA2 staining is absent in nuclei of the goblet cells (C—H). Figures are representative of at least 4 individual mice of each genotype (bar=200 μm).

FIG. 6. Timed deletion of FoxA2 during lung morphogenesis. The period of doxycycline treatment is shown in the arrows above. Lung sections from FoxA2-rtTA-/tg, (tetO)7CMV-Cre-/tg, FoxA2loxP/loxP (B, D, F, H) and littermate controls (A, C, E, G) are prepared on PN16 and stained with hematoxylin-eosin. Airspace enlargement and neutrophilic infiltrations are observed in the FoxA2 deleted mice. Figures are representative of n=4 (bar=300 μm).

FIG. 7. FOXA1, FOXJ1, PECAM, and elastin staining. Lung sections are prepared on PN16. Triple transgenic mice, FoxA2-rtTA-/tg, (tetO)7CMV-Cre-/tg, FoxA2loxP/loxP and littermate controls are maintained on doxycycline from E0. Lung sections are stained for FOXA1 (A, B), FOXJ1 (C, D), PECAM (E, F), and elastin (G, H). Inserts are the larger magnification (×40) of corresponding figures. Figures are representative of n=4 (bar=10 μm).

FIG. 8. Pulmonary mechanics. (A) Airway resistance, (B) airway elastance, (C) compliance, (D) tissue damping, (E) tissue elastance, and (F) hysteresivity are measured in 7-week old CCSP-rtTA, FoxA2Δ/Δ (closed bar, n=6) and control mice (open bar, n=5). Values are expressed as mean±s.e.m. Increased airway resistance, airway elastance, tissue damping, tissue elastance, and decreased compliance are observed. *P<0.05 vs. control mice (by Student's t test).

FIG. 9. FoxA2 staining of mouse models with goblet cell hyperplasia. Lung sections are prepared and stained for FoxA2 from mouse models with goblet cell hyperplasia: over-expression of IL-4 (A, B, C), IL-13 (D, E, F), deletion of FoxA2 (G, H, I), and ovalbumin challenged mice (J, K, L). Normal bronchi (A, D, G, J), and bronchi with goblet cell hyperplasia (B, E, H, K) are shown. FoxA2/Alcian blue staining (C, F, I, L) indicates a close correlation between goblet cell hyperplasia and decreased or absent FoxA2 expression. Figures are representative of n=4 (bar=50 μm).

FIG. 10. Effects of IL-4 on FoxA2 are STAT-6 dependent. Adult control (A, B, C, D) and Stat-6−/− (E, F, G, H) mice are treated intranasally with IL-4. Lung sections are prepared and stained for FoxA2. The mice are treated with saline (A, B, E, F) or IL-4 (C, D, G, H). IL-4 induced goblet cell hyperplasia in control but not Stat-6−/− mice. FoxA2 staining is decreased at sites of goblet cell hyperplasia in wild type mice. FoxA2 staining and epithelial cell morphology are not perturbed in the Stat-6−/− mice. Figures are representative of n=4 mice of each genotype, bar=50 μm.

FIG. 11. FoxA2 inhibits MUC5A/C transcription. H292 cells are transfected with promoter plasmid pGL3-MUC5A/C-luc and increasing amounts of the expression plasmid pRC-CMV-FoxA2. MUC5A/C promoter activity is determined by relative luciferase activity normalized to β-galactosidase activity. All transretinoic acid 3 μg/ml is added to induce MUC5A/C expression as positive control. Plasmid pRC-CMV is used as empty vector control. *P<0.05 vs. control (by ANOVA).

FIG. 12. FoxA2 staining of human lung tissue with goblet cell hyperplasia. A. Photomicrograph (×40) of a small conducting airway of a 5-month-old infant who died with bronchopulmonary dysplasia. The Alcian blue stain mucus cells have nuclei unstained by FoxA2 antibody. A few cells with FoxA2 stained nuclei are noted in the basal layer of the conducting airways and a nearby terminal airway. B. Photomicrograph (×10) of the lung from the same patient seen in panel A, showing a cuboidal lined alveolar duct arising from the small columnar cell lined bronchus. The nuclei of the Alcian blue stained mucus cells in the bronchus lack FoxA2, but most of the nuclei of the cells lining the alveolar ducts are FoxA2 reactive. C. Photomicrograph (×40) of the lung of a 27-year-old female who underwent lobectomy for bronchiectasis. This shows a small bronchus lined with Alcian blue stained mucus cells, the nuclei of which lack FoxA2 staining. Many nuclei of non-goblet cells lining terminal airways are FoxA2 reactive. D. Photomicrograph (×10) of the lung of a 6 ½-month-old infant dying with bronchopulmonary dysplasia showing terminal airway cell nuclei immunostained for FoxA2. Nuclei of epithelial cells lining a small conducting airway lack Alcian blue staining and are immunostained by the FoxA2 antibody. Bar=50 μm.

FIG. 13. Western blot demonstrate decreased SP-B expression. FoxA2 compound mice and their littermate controls are maintained on doxycycline from E0. Lung homogenates are prepared at PN16. Equal amount of proteins are analyzed by Western blot using SP-B or FoxA2 rabbit polyclonal antibodies. Protein quantity is analyzed by ImageQuant software. Control values are arbitrarily set to 100%. *P<0.05 vs. control mice (by Student's t test).

FIG. 14. Goblet cells in small and big airways of FoxA2Δ/Δ mice. Lung sections of PN16 FoxA2 (A, C) and CCSP (B, D) compound mice are stained for FoxA2. Goblet cells are found in small (A, B) and big airways (C, D) of FoxA2Δ/Δ mice. (bar=100 μm).

FIG. 15. FOXA1, FOXJ1, PECAM, and elastin staining. Lung sections are prepared on PN16. Triple transgenic mice, FoxA2-rtTA-/tg, (tetO)7CMV-Cre-/tg, FoxA2loxP/loxP and littermate controls are maintained on doxycycline from E0. Lung sections are stained for FOXA1 (A, B), FOXJ1 (C, D), PECAM (E, F), and elastin (G, H). Inserts are the larger magnification (×40) of corresponding figures. Figures are representative of n=4 (bar=100 μm).

[symbol]

### DETAILED DESCRIPTION OF THE INVENTION

Before the present proteins, nucleotide sequences, and methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All references, publications, patents, patent applications, and commercial materials mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention. In order to provide a clear and consistent understanding of the specification and claims, including the scope to be given such terms, the following definitions are provided.

The term “agonist”, as used herein, is meant to refer to an agent that mimics or upregulates (e.g. potentiates or supplements) FoxA2 bioactivity. A FoxA2 agonist can be a wild-type FoxA2 protein or derivative thereof having at least one bioactivity of the wild-type FoxA2. A FoxA2 therapeutic can also be a compound that upregulates expression of a FoxA2 gene or which increases at least one bioactivity of the FoxA2 protein. Agonists can be any class of molecule, preferably a small molecule, including a nucleic acid, protein, carbohydrate, lipid or combination thereof.

“Antagonist” as used herein is meant to refer to an agent that down-regulates (e.g. suppresses or inhibits) at least one FoxA2 bioactivity. An antagonist can be a compound that down-regulates expression of a FoxA2 locus gene or that reduces the amount of a FoxA2 protein present. The FoxA2 antagonist can also be a FoxA2 antisense nucleic acid or a ribozyme capable of interacting specifically with FoxA2 RNA. Yet other FoxA2 antagonists are molecules that bind to FoxA2 polypeptide and inhibit its action. Such molecules include peptides. Yet other FoxA2 antagonists include antibodies interacting specifically with an epitope of a FoxA2 molecule, such that binding interferes with the biological function of the FoxA2 locus polypeptide.

The term “antisense”, as used herein, refers to nucleotide sequences which are complementary to a specific DNA or RNA sequence. The term “antisense strand” is used in reference to a nucleic acid strand that is complementary to the “sense” strand. Antisense molecules may be produced by any method, including synthesis by ligating the gene(s) of interest in a reverse orientation to a viral promoter, which permits the synthesis of a complementary strand. Once introduced into a cell, this transcribed strand combines with natural sequences produced by the cell to form duplexes. These duplexes then block either the further transcription or translation. In this manner, mutant phenotypes may be generated. The designation “negative” is sometimes used in reference to the antisense strand, and “positive” is sometimes used in reference to the sense strand.

“Biological activity” or “bioactivity” or “activity” or “biological function”, which are used interchangeably, for the purposes herein means a function that is directly or indirectly performed by a FoxA2 polypeptide (whether in its native or denatured conformation), or by any subsequence thereof. FoxA2 bioactivity can be modulated by directly affecting a FoxA2 polypeptide. Alternatively, a FoxA2 bioactivity can be modulated by modulating the level of a FoxA2 polypeptide, such as by modulating expression of a FoxA2 gene.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG), and PLURONICS.

The term “cDNA” as used herein is intended to include all nucleic acids that share the arrangement of sequence elements found in native mature mRNA species, where sequence elements are exons, 3′ and 5′ non-coding regions. Normally mRNA species have contiguous exons, with the intervening introns deleted, to create a continuous open reading frame encoding FoxA2.

“Cells”, “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A “chimeric gene” refers to a sequence of DNA in which nucleotide sequences not naturally occurring together are linked. As used to describe this aspect of the invention, the term “chimeric” requires that the amino acid sequence of the chimeric molecule include at least one stretch of amino acids (preferably stretches of 50-300, or more amino acids) from the naturally-occurring polypeptide from which it was derived. Thus, the chimeric polypeptide is a “hybrid” or “mosaic” of two or more polypeptides. By “chimeric” is meant that the polypeptide of the invention is not identical to any naturally occurring polypeptide sequence (or fragment of a natural polypeptide sequence).

A “chimeric polypeptide” or “fusion polypeptide” is a fusion of a first amino acid sequence encoding one of the subject FoxA2 locus polypeptides with a second amino acid sequence defining a domain (e.g. polypeptide portion) foreign to and not substantially homologous with any domain of a FoxA2 polypeptide. A chimeric polypeptide may present a foreign domain that is found (albeit in a different polypeptide) in an organism that also expresses the first polypeptide, or it may be an “interspecies”, “intergenic”, etc. fusion of polypeptide structures expressed by different kinds of organisms. In general, a fusion polypeptide can be represented by the general formula X-FoxA2-Y, wherein FoxA2 represents a portion of the polypeptide that is derived from a FoxA2 polypeptide, and X and Y are independently absent or represent amino acid sequences that are not related to a FoxA2 sequence in an organism, including naturally occurring mutants.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

The term “chronic bronchitis” refers to the condition associated with prolonged exposure to nonspecific bronchial irritants and is accompanied by mucus hypersecretion and structural changes in the bronchi.

The term “chronic obstructive bronchitis” means the disease condition frequently associated with the symptoms of chronic bronchitis in which disease of the small airways has progressed to the point that there is clinically significant airway obstruction.

The term “chronic asthmatic bronchitis” refers to an underlying asthmatic condition in patients in whom asthma has become so persistent that clinically significant chronic airflow obstruction is present despite antiasthmatic therapy.

The term “chronic obstructive pulmonary disease or disorder”, or “COPD”, is defined as a generally progressive disease state, due to chronic obstructive bronchitis or chronic obstructive emphysema, which may be accompanied by airway hyperreactivity and may be partially reversible.

A “clone” is a population of cells derived from a single cell or common ancestor by mitosis. A “cell line” is a derivative of a primary cell culture that is capable of stable growth in vitro for many generations.

A DNA “coding sequence” or a “nucleotide sequence encoding” a particular protein, is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, procaryotic sequences, cDNA from eucaryotic mRNA, genomic DNA sequences from eucaryotic (e.g., mammalian) DNA, and even synthetic DNA sequences. A transcription termination sequence will usually be located 3′ to the coding sequence.

The terms “complementary” or “complementarity”, as used herein, refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between the single stranded molecules. The degree of complementarity between nucleic acid strands has significant effects on the efficiency and strength of hybridization between nucleic acid strands. This is of particular importance in amplification reactions, which depend upon binding between nucleic acids strands.

“Consensus”, as used herein, refers to a nucleic acid sequence which has been re-sequenced to resolve uncalled bases, or which has been extended using XL-PCR (Perkin Elmer, Norwalk, Conn.) in the 5′ and/or the 3′ direction and re-sequenced, or which has been assembled from the overlapping sequences of more than one Incyte clone using the GELVIEW Fragment Assembly system (GCG, Madison, Wis.), or which has been both extended and assembled.

DNA “control sequences” refer collectively to promoter sequences, ribosome binding sites, polyadenylation signals, transcription termination sequences, upstream regulatory domains, enhancers, and the like, which collectively provide for the transcription and translation of a coding sequence in a host cell.

A “covalent bond” is defined as the formation of a sigma bond between two organic molecules. A “non-covalent bond” is meant to include all interactions other than a covalent bond. Non-covalent bonds include ionic interactions, hydrogen bonding, pi-pi bonding, hydrophobic interactions, and van der Waals interactions.

A “deletion”, as used herein, refers to a change in either amino acid or nucleotide sequence in which one or more amino acid or nucleotide residues, respectively, are absent.

“Deoxyribonucleic Acid (DNA)” is the molecular basis of heredity. DNA consists of a polysugar-phosphate backbone from which the purines and pyrimidines project. Bonds between the phosphate molecule and carbon 3 and carbon 5 of adjacent deoxyribose molecules form the backbone. The nitrogenous base extends from carbon 1 of each sugar. According to the Watson-Crick model, DNA forms a double helix that is held together by hydrogen bonds between specific pairs of bases (thymine to adenine and cytosine to guanine). Each strand in the double helix is complementary to its partner strand in terms of its base sequence.

The term “derivative”, as used herein, refers to the chemical modification of a nucleic acid. Illustrative of such modifications would be replacement of hydrogen by an alkyl, acyl, or amino group. A nucleic acid derivative would encode a polypeptide, which retains essential biological characteristics of the natural molecule.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. After digestion, the reaction is electrophoresed directly on an agarose or polyacrylamide gel to isolate the desired fragment.

The phrases “disruption of the gene” and “targeted disruption” or any similar phrase refers to the site specific interruption of a native DNA sequence so as to prevent expression of that gene in the cell as compared to the wild-type copy of the gene. The interruption may be caused by deletions, insertions or modifications to the gene, or any combination thereof.

A “DNA construct” is a DNA molecule, or a clone of such a molecule, either single- or double-stranded that has been modified through human intervention to contain segments of DNA combined and juxtaposed in a manner that as a whole would not otherwise exist in nature.

As is well known, genes may exist in single or multiple copies within the genome of an individual. Such duplicate genes may be identical or may have certain modifications, including nucleotide substitutions, additions or deletions, which still code for polypeptides having substantially the same activity. The term “DNA sequence encoding a FoxA2 polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a polypeptide with the same biological activity.

A “double-stranded DNA molecule” refers to the polymeric form of deoxyribonucleotides (bases adenine, guanine, thymine, or cytosine) in a double-stranded helix, both relaxed and supercoiled. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having the sequence homologous to the mRNA).

An “effective amount” or “therapeutically effective amount” of an active agent disclosed herein is an amount capable of modulating, to some extent, the activity of a target cell and preferably is an amount capable of modulating, to some extent, the growth or activity of a target cell. The term includes an amount capable of invoking an inhibitory effect on goblet cell characterization and products (e.g., mucous, inflammation, etc.) effect of the target cells. An “effective amount” may be determined empirically and in a routine manner. An “effective amount” of a polypeptide disclosed herein or an antagonist thereof, in reference to inhibition of mucous gene transcription is an amount capable of inhibiting, to some extent, mucous production. The term includes an amount capable of invoking an effect on cell differentiation of the target cells. Administration “in combination with” one or more further therapeutic agents includes simultaneous (concurrent) and consecutive administration in any order.

The term “encoding” refers generally to the sequence information being present in a translatable form, usually operably linked to a promoter. A sequence is operably linked to a promoter when the functional promoter enhances transcription or expression of that sequence. An anti-sense strand is considered to also encode the sequence, since the same informational content is present in a readily accessible form, especially when linked to a sequence that promotes expression of the sense strand. The information is convertible using the standard, or a modified, genetic code. See, e.g., Watson et al., (1987) The Molecular Biology of the Gene (4th ed.) vols. 1&2, Benjamin, Menlo Park, Calif.

“Filling” or “blunting” refers to the procedures by which the single stranded end in the cohesive terminus of a restriction enzyme-cleaved nucleic acid is converted to a double strand. This eliminates the cohesive terminus and forms a blunt end. This process is a versatile tool for converting a restriction cut end that may be cohesive with the ends created by only one or a few other restriction enzymes into a terminus compatible with any blunt-cutting restriction endonuclease or other filled cohesive terminus. Typically, blunting is accomplished by incubating 2-15 μg of the target DNA in 10 mM MgCl₂, 1 mM dithiothreitol, 50 mM NaCl, 10 mM Tris (pH 7.5) buffer at about 37° C. in the presence of 8 units of the Klenow fragment of DNA polymerase I and 250 μM of each of the four deoxynucleoside triphosphates. The incubation generally is terminated after 30 min. phenol and chloroform extraction and ethanol precipitation.

As used herein, the term “FK506 analogs” refers to compounds that are functionally analogous to FK506 in their ability to stimulate neuritic outgrowth, such as V-10,367 (Armistead et al., Acta Crystallogr. 51:522-528, 1995). See, for example: Bierer et al., Science 250:556-559, 1990; Van Duyne et al., Science 252:839-842, 1991; Van Duyne et al., J. Mol. Biol. 229:105-124, 1993; Hauske et al., J. Med. Chem. 35:4284-4296, 1992; Holt et al., J. Am. Chem. Soc. 115:9925-9938, 1993; Holt et al., Bioorg. Med. Chem. Lett. 3:1977-1980, 1993; Teague and Stocks, Bioorg. Med. Chem. Lett. 3:1947-1950, 1993; Wang et al., Bioorg. Med. Chem. Lett. 4:1161-1166, 1994; Yamashita et al., Bioorg. Med. Chem. Lett. 4:325-328, 1994; Stocks et al., Bioorg. Med. Chem. Lett. 4:1457-1460, 1994; Goulet et al., Perspect. Drug Disc. Design 2:145-162, 1994; Wilson et al., Acta Cryst. D51:511-S21, 1995; Armistead et al., Acta Cryst. D51:522-528, 1995; U.S. Pat. Nos. 5,192,773, 5,330,993, 5,516,797, 5,612,350, 5,614,547, 5,622,970, 5,654,332; and published international patent applications WO 92/00278, WO 92/04370, WO 92/19593, WO 92/21313, WO 94/07858, and WO 96/40633. FK506 analogs include, but are not limited to compounds described in U.S. Pat. Nos. 5,622,970, 5,516,797, 5,330,993, 5,192,773, and WO 92/00278.

Where the term “FoxA2” is used in reference to a gene product or polypeptide, it is meant to refer to all gene products encoded by the FoxA2 locus on human chromosome 20 and their corresponding mouse homologs.

The DNA sequence encoding FoxA2 may be cDNA or genomic DNA or a fragment thereof. The term “FoxA2 gene” means the open reading frame encoding specific FoxA2 polypeptides, as well as adjacent 5′ and 3′ non-coding nucleotide sequences involved in the regulation of expression, up to about 1 kb beyond the coding region, in either direction. The gene may be introduced into an appropriate vector for extrachromosomal maintenance or for integration into the host.

“FoxA2 Locus,” “FoxA2 Gene,” “FoxA2 Nucleic Acids” or “FoxA2 Polynucleotide” each refer to polynucleotides, all of which are in the FoxA2 region, that are likely to be expressed in normal tissue. The FoxA2 locus is intended to coding sequences, intervening sequences and regulatory elements controlling transcription and/or translation. The FoxA2 locus is intended to include all allelic variations of the DNA sequence. These terms, when applied to a nucleic acid, refer to a nucleic acid that encodes a FoxA2 polypeptide, fragment, homolog or variant, including, e.g., protein fusions or deletions. The nucleic acids of the present invention will possess a sequence that is either derived from, or substantially similar to a natural FoxA2-encoding gene or one having substantial homology with a natural FoxA2-encoding gene or a portion thereof. The coding sequence for a human FoxA2 polypeptide is shown in SEQ ID NO:1 (variant 1) and SEQ ID NO:3 (variant 2), with the amino acid sequence shown in SEQ ID NO:2.

“FoxA2 protein” or “FoxA2 polypeptide” refer to a protein or polypeptide encoded by the FoxA2 locus, variants or fragments thereof. The term “polypeptide” refers to a polymer of amino acids and its equivalent and does not refer to a specific length of the product; thus, peptides, oligopeptides and proteins are included within the definition of a polypeptide. This term also does not refer to, or exclude modifications of the polypeptide, for example, glycosylations, acetylations, phosphorylations, and the like. Included within the definition are, for example, polypeptides containing one or more analogs of an amino acid (including, for example, unnatural amino acids, etc.), polypeptides with substituted linkages as well as other modifications known in the art, both naturally and non-naturally occurring. Ordinarily, such polypeptides will be at least about 50% homologous to the native FoxA2 sequence, preferably in excess of about 90%, and more preferably at least about 95% homologous. Also included are proteins encoded by DNA that hybridize under high or low stringency conditions, to FoxA2-encoding nucleic acids and closely related polypeptides or proteins retrieved by antisera to the FoxA2 protein(s). The FoxA2 polypeptide of the present invention also includes conservative variations of the polypeptide sequence. The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine glutamic for aspartic acids, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

“FoxA2 Region” refers to a portion of human chromosome 20. This region contains the FoxA2 locus, including the FoxA2 gene. As used herein, the terms “FoxA2 locus,” “FoxA2 allele” and “FoxA2 region” all refer to the double-stranded DNA comprising the locus, allele, or region, as well as either of the single-stranded DNAs comprising the locus, allele or region. As used herein, a “portion” of the FoxA2 locus or region or allele is defined as having a minimal size of at least about 50, 100, 150, 200, 250, or 300 nucleotides and preferably have a minimal size of at least about 400 nucleotides.

The term “FoxA2 related” as used herein is meant to include all mouse and human genes related to the human FoxA2 locus genes on human chromosome 20.

The term “FoxA2 therapeutic” refers to various forms of FoxA2 polypeptides, as well as peptidomimetics, nucleic acids, or small molecules, which can modulate at least one activity of a FoxA2 polypeptide by mimicking or potentiating (agonizing) or inhibiting (antagonizing) the effects of a naturally-occurring FoxA2 polypeptide. A FoxA2 therapeutic that mimics or potentiates the activity of a wild-type FoxA2 polypeptide is a “FoxA2 agonist”. Conversely, a FoxA2 therapeutic that inhibits the activity of a wild-type FoxA2 polypeptide is an “FoxA2 antagonist”.

The term “gene” means the segment of DNA involved in producing a polypeptide chain; it includes regions preceding and following the coding region “leader and trailer” as well as intervening sequences (introns) between individual coding segments (exons).

The phrases “gene amplification” and “gene duplication” are used interchangeably and refer to a process by which multiple copies of a gene or gene fragment are formed in a particular cell or cell line. The duplicated region (a stretch of amplified DNA) is often referred to as “amplicon.” Usually, the amount of the messenger RNA (mRNA) produced, i.e., the level of gene expression, also increases in the proportion of the number of copies made of the particular gene expressed.

A “gene fusion” is a DNA construction (performed in vitro or in vivo) that results in the coding sequences from one gene (the “responder”) being transcribed and/or translated under the direction of the controlling sequences of another gene (the “controller”). Responder genes can be divided into two classes, reporters and effectors, with analytical or manipulative roles, respectively.

The term “haplotype” refers to a set of alleles that are inherited together as a group (are in linkage disequilibrium). As used herein, haplotype is defined to include those haplotypes that occur at statistically significant levels (p<0.05). As used herein, the phrase “a FoxA2 haplotype” refers to a haplotype in the FoxA2 locus.

The term “heterozygote” “heterozygotic mammal” and the like, refers to a transgenic mammal with a knockout construct on one of a chromosome pair in all of its genome-containing cells.

The term “homology”, as used herein, refers to a degree of complementarity. There may be partial homology or complete homology (i.e., identity). A partially complementary sequence is one that at least partially inhibits an identical sequence from hybridizing to a target nucleic acid; it is referred to using the functional term “substantially homologous.” The inhibition of hybridization of the completely complementary sequence to the target sequence may be examined using a hybridization assay (Southern or northern blot, solution hybridization and the like) under conditions of low stringency. A substantially homologous sequence or probe will compete for and inhibit the binding (i.e., the hybridization) of a completely homologous sequence or probe to the target sequence under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target sequence which lacks even a partial degree of complementarity (e.g., less than about 30% identity); in the absence of non-specific binding, the probe will not hybridize to the second non-complementary target sequence. An “unrelated” or “non-homologous” sequence shares less than 40% identity, though preferably less than 25% identity, with one of the FoxA2 locus sequences of the present invention. The terms “substantial homology” or “substantial identity”, when referring to polypeptides, indicate that the polypeptide or protein in question exhibits at least about 30% identity with an entire naturally-occurring protein or a portion thereof, usually at least about 70% identity, and preferably at least about 95% identity. “Substantially similar function” refers to the function of a modified nucleic acid or a modified protein, with reference to the wild-type FoxA2 nucleic acid or wild-type FoxA2 polypeptide. The modified polypeptide will be substantially homologous to the wild-type FoxA2 polypeptide and will have substantially the same function. The modified polypeptide may have an altered amino acid sequence and/or may contain modified amino acids. In addition to the similarity of function, the modified polypeptide may have other useful properties, such as a longer half-life. The similarity of function (activity) of the modified polypeptide may be substantially the same as the activity of the wild-type FoxA2 polypeptide. Alternatively, the similarity of function (activity) of the modified polypeptide may be higher than the activity of the wild-type FoxA2 polypeptide. The modified polypeptide is synthesized using conventional techniques, or is encoded by a modified nucleic acid and produced using conventional techniques. The modified nucleic acid is prepared by conventional techniques. A nucleic acid with a function substantially similar to the wild-type FoxA2 gene function produces the modified protein described above. Homology, for polypeptides, is typically measured using sequence analysis software. Protein analysis software matches similar sequences using measure of homology assigned to various substitutions, deletions and other modifications. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. “Substantial homology or similarity”. A nucleic acid or fragment thereof is “substantially homologous” (“or substantially similar”) to another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases.

Alternatively, substantial homology or (similarity) exists when a nucleic acid or fragment thereof will hybridize to another nucleic acid (or a complementary strand thereof) under selective hybridization conditions, to a strand, or to its complement. Selectivity of hybridization exists when hybridization that is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides. Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. The preparation of such probes and suitable hybridization conditions are well known in the art.

The term “homozygote” “homozygotic mammal” and the like, refers to a transgenic mammal with a knockout construct on both members of a chromosome pair in all of its genome-containing cells.

A “host cell” is a cell that has been transformed, or is capable of transformation, by an exogenous DNA sequence.

The term “hybridization”, as used herein, refers to any process by which a strand of nucleic acid binds with a complementary strand through base pairing.

The term “hybridization complex”, as used herein, refers to a complex formed between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an antiparallel configuration.

The term “intron” identifies an intervening sequence within a gene for the gene product that does not constitute protein coding sequences. In eukaryotic cells introns are removed from the primary RNA transcript to produce the mature mRNA.

An “insertion” or “addition”, as used herein, refers to a change in an amino acid or nucleotide sequence resulting in the addition of one or more amino acid or nucleotide residues, respectively, as compared to the naturally occurring molecule.

The term “interact” as used herein is meant to include detectable relationships or association (e.g. biochemical interactions) between molecules, such as interaction between protein-protein, protein-nucleic acid, nucleic acid-nucleic acid, and protein-small molecule or nucleic acid-small molecule in nature.

An “isolated” nucleic acid is a nucleic acid, e.g., an RNA, DNA, or a mixed polymer, which is substantially separated from other DNA sequences which naturally accompany a native human sequence, e.g., ribosomes, polymerases, and many other human genome sequences. The term embraces a nucleic acid sequence that has been removed from its naturally occurring environment, and includes recombinant or cloned DNA isolates and chemically synthesized analogs or analogs biologically synthesized by heterologous systems. A substantially pure molecule includes isolated forms of the molecule. An isolated nucleic acid will generally be a homogenous composition of molecules, but will, in some embodiments, contain minor heterogeneity. This heterogeneity is typically found at the polymer ends or portions not critical to a desired biological function or activity.

The word “label” when used herein refers to a detectable compound or composition which is conjugated directly or indirectly to the peptide or nucleotide so as to generate a “labeled” entity. The label may be detectable by itself (e.g. radioisotope labels or fluorescent labels) or, in the case of an enzymatic label, may catalyze chemical alteration of a substrate compound or composition, which is detectable.

A “ligand” is a molecule, other than an antibody or an immunoglobulin, capable of being bound by the ligand-binding domain of a receptor. The molecule may be chemically synthesized or may occur in nature.

“Ligation” refers to the process of forming phosphodiester bonds between two double stranded nucleic acid fragments. Unless otherwise provided, ligation is accomplished using known buffers and conditions with T4 DNA ligase (“ligase”) and approximately equimolar amounts of the DNA fragments to be ligated.

A “liposome” is a small vesicle composed of various types of lipids, phospholipids and/or surfactant which is useful for delivery of a drug (such as an active agent, herein) to a mammal. The components of the liposome are commonly arranged in a bilayer formation, similar to the lipid arrangement of biological membranes.

The term “maintained” refers to the stable presence of a plasmid within a transformed host cell wherein the plasmid is present as an autonomously replicating body or as an integrated portion of the host genome.

“Mammal” or “subject” for purposes of treatment refers to any animal classified as a mammal, including humans, domestic and farm animals, and zoo, sports, or pet animals, such as dogs, cats, cattle, horses, sheep, pigs, goats, rabbits, etc. Preferably, the mammal is human.

The term “marker” or “marker sequence” or similar phrase means any gene that produces a selectable genotype or preferably a selectable phenotype. It includes such examples as the neo gene, green fluorescent protein (GFP) gene, TK gene, β-galactosidase gene, etc. The marker sequence may be any sequence known to those skilled in the art that serves these purposes, although typically the marker sequence will be a sequence encoding a protein that confers a selectable trait, such as an antibiotic resistance gene, or an enzyme that can be detected and that is not typically found in the cell. The marker sequence may also include regulatory regions such as a promoter or enhancer that regulates the expression of that protein. However, it is also possible to transcribe the marker using endogenous regulatory sequences. In one embodiment of the present invention, the marker facilitates separation of transfected from untransfected cells by fluorescence activated cell sorting, for example by the use of a fluorescently labeled antibody or the expression of a fluorescent protein such as GFP. Other DNA sequences that facilitate expression of marker genes may also be incorporated into the DNA constructs of the present invention. These sequences include, but are not limited to transcription initiation and termination signals, translation signals, post-translational modification signals, intron splicing junctions, ribosome binding sites, and polyadenylation signals, to name a few. The marker sequence may also be used to append sequence to the target gene. For example, it may be used to add a stop codon to truncate FoxA2 translation.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “modulate”, as used herein, refers to a change or an alteration in the biological activity of FoxA1 and/or FoxA2. Modulation may be an increase or a decrease in protein activity, a change in binding characteristics, or any other change in the biological, functional or other properties of a Fox A1 and/or FoxA2 proteins.

The use of selectable markers is well known in the art and need not be detailed herein. The term “modulation” as used herein refers to both upregulation (i.e., activation or stimulation (e.g., by agonizing or potentiating)) and downregulation (i.e. inhibition or suppression (e.g., by antagonizing, decreasing or inhibiting)).

The term “mutated gene” refers to an allelic form of a gene, which is capable of altering the phenotype of a subject having the mutated gene relative to a subject that does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous subject (for that gene), the mutation is said to be co-dominant.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs and as applicable to the embodiment being described, single (sense or antisense) and double-stranded polynucleotides.

The phrase “nucleotide sequence complementary to the nucleotide sequence set forth in SEQ ID NO. x” refers to the nucleotide sequence of the complementary strand of a nucleic acid strand having SEQ ID NO. x. The term “complementary strand” is used herein interchangeably with the term “complement”. The complement of a nucleic acid strand can be the complement of a coding strand or the complement of a non-coding strand. When referring to double stranded nucleic acids, the complement of a nucleic acid having SEQ ID NO. x refers to the complementary strand of the strand having SEQ ID NO. x or to any nucleic acid having the nucleotide sequence of the complementary strand of SEQ ID NO. x. When referring to a single stranded nucleic acid having the nucleotide sequence SEQ ID NO. x, the complement of this nucleic acid is a nucleic acid having a nucleotide sequence which is complementary to that of SEQ ID NO. x. The nucleotide sequences and complementary sequences thereof are always given in the 5′ to 3′ direction.

“Oligonucleotides” refers to either a single stranded polydeoxynucleotide or two complementary polydeoxynucleotide strands, which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The term “open reading frame” refers to a nucleotide sequence with the potential for encoding a protein.

“Operably linked” refers to a juxtaposition wherein the components so described are in a relationship permitting them to function in their intended manner. For instance, a promoter is operably linked to a coding sequence if the promoter affects its transcription or expression.

The term “polypeptide” refers to a polymer of amino acids and its equivalent and does not refer to a specific length of the product; thus, peptides, oligopeptides and proteins are included within the definition of a polypeptide. This term also does not exclude modifications of the polypeptide, for example, glycosylations, acetylations, phosphorylations, and the like. Included within the definition are, for example, polypeptides containing one or more analogs of an amino acid (including, for example, unnatural amino acids, etc.), polypeptides with substituted linkages as well as other modifications known in the art, both naturally and non-naturally occurring.

A “polypeptide variant” of any one of the polypeptides will have at least about 80% amino acid sequence identity, preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and most preferably at least about 99% amino acid sequence identity with a full-length native sequence as disclosed herein, such variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 300 amino acids in length, or more.

The term “prodrug” as used in this application refers to a precursor or derivative form of a pharmaceutically active substance that is less cytotoxic to tumor cells compared to the parent drug and is capable of being enzymatically activated or converted into the more active parent form. The prodrugs of this invention include, but are not limited to, phosphate-containing prodrugs, thiophosphate-containing prodrugs, sulfate-containing prodrugs, peptide-containing prodrugs, D-amino acid-modified prodrugs, glysocylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug.

As used herein, the term “promoter” means a DNA sequence that regulates expression of a selected DNA sequence operably linked to the promoter, and which effects expression of the selected DNA sequence in cells. The term encompasses “tissue specific” promoters, i.e. promoters, which effect expression of the selected DNA sequence only in specific cells (e.g. cells of a specific tissue). The term also covers so-called “leaky” promoters, which regulate expression of a selected DNA primarily in one tissue, but cause expression in other tissues as well. The term also encompasses non-tissue specific promoters and promoters that constitutively express or that are inducible (i.e. expression levels can be controlled).

A “promoter sequence” is a DNA regulatory region capable of binding RNA polymerase in a cell and initiating transcription of a downstream (3′ direction) coding sequence. For purposes of defining the present invention, the promoter sequence is bound at the 3′ terminus by the translation start codon (ATG) of a coding sequence and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase.

The term “protein” is used herein to designate a naturally occurring polypeptide. The term “polypeptide” is used in its broadest sense, i.e., any polymer of amino acids (dipeptide or greater) linked through peptide bonds. Thus, the term “polypeptide” includes proteins, oligopeptides, protein fragments, analogs, muteins, fusion proteins and the like. “Native” proteins or polypeptides refer to proteins or polypeptides recovered from a source occurring in nature. The terms “protein”, “polypeptide” and “peptide” are used interchangeably herein when referring to a gene product. “Protein modifications or fragments” are provided by the present invention for FoxA2 polypeptides or fragments thereof which are substantially homologous to primary structural sequence but which include, e.g., in vivo or in vitro chemical and biochemical modifications or which incorporate unusual amino acids. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as ³²P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods of labeling polypeptides are well known in the art. Besides substantially full-length polypeptides, the present invention provides for biologically active fragments of the polypeptides or modifications of the polypeptides that could improve efficacy. Significant biological activities include ligand-binding, immunological activity and other biological activities characteristic of FoxA2 polypeptides.

As used herein, “pulmonary disease” refers to disorders and conditions generally recognized by those skilled in the art as related to the constellation of pulmonary diseases characterized by emphysema, monocytic infiltrates, fibrosis, epithelial cell dysplasia, and atypical accumulations of intracellular lipids in type II epithelial cells and alveolar macrophages, regardless of the cause or etiology. These include, but are not limited “airway obstructive diseases” e.g., respiratory disorder, such as, airway obstruction, allergies, asthma, acute inflammatory lung disease, chronic inflammatory lung disease, chronic obstructive pulmonary dysplasia, emphysema, pulmonary emphysema, chronic obstructive emphysema, adult respiratory distress syndrome, bronchitis, chronic bronchitis, chronic asthmatic bronchitis, chronic obstructive bronchitis, and intestitial lung diseases.

The term “pulmonary emphysema” refers to enlargement of the airspaces distal to the terminal nonrespiratory bronchioles, accompanied by destructive changes of the alveolar walls. The term “chronic obstructive emphysema” refers to the condition when there has been sufficient loss of lung recoil to allow marked airway collapse upon expiration, leading to the physiologic pattern of airway obstruction.

The term “recombinant” refers to a nucleic acid sequence that is not naturally occurring, or is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the common natural forms. Restriction enzyme recognition sites are often the target of such artificial manipulations, but other site-specific targets, e.g., promoters, DNA replication sites, regulation sequences, control sequences, or other useful features may be incorporated by design. A similar concept is intended for a recombinant, e.g., fusion, polypeptide.

The term “recombinant protein” refers to a polypeptide of the present invention which is produced by recombinant DNA techniques, wherein generally, DNA encoding a FoxA2 polypeptide is inserted into a suitable expression vector which is in turn used to transform a host cell to produce the heterologous protein. Moreover, the phrase “derived from”, with respect to a recombinant FoxA2 gene, is meant to include within the meaning of “recombinant protein” those proteins having an amino acid sequence of a native FoxA2 polypeptide, or an amino acid sequence similar thereto which is generated by mutations including substitutions and deletions (including truncation) of a naturally occurring form of the polypeptide.

“Regulatory sequences” refers to those sequences normally within 100 kb of the coding region of a locus, but they may also be more distant from the coding region, which affect the expression of the gene (including transcription of the gene, and translation, splicing, stability or the like of the messenger RNA).

A “replicon” is any genetic element (e.g., plasmid, chromosome, virus) that functions as an autonomous unit of DNA replication in vivo; i.e., capable of replication under its own control.

The term “signal peptide” refers to any peptide sequence that directs a polypeptide to which it is attached to a target cell and, preferably, directs its transport across the cell membrane. An “importation competent signal peptide sequence” is one that remains competent to translocate the attached peptide sequence across a cellular membrane.

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention to identify compounds that modulate a FoxA2 bioactivity.

As used herein, the term “specifically hybridizes” or “specifically detects” refers to the ability of a nucleic acid molecule of the invention to hybridize to at least approximately 6, 12, 20, 30, 50, 100, 150, 200, 300, 350, 400 or 425 consecutive nucleotides of a vertebrate, preferably a FoxA2 gene.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to re-anneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so.

A “substitution”, as used herein, refers to the replacement of one or more amino acids or nucleotides by different amino acids or nucleotides, respectively.

A “therapeutically effective amount”, in reference to the treatment of tumor, refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder.

“Transcriptional fusions” are gene fusions in which all coding sequences are derived from the responder gene. “Translational fusions” are gene fusions that encode a polypeptide comprising coding information of the controller and responder genes.

“Transcriptional regulatory sequence” is a generic term used throughout the specification to refer to DNA sequences, such as initiation signals, enhancers, and promoters, which induce or control transcription of protein coding sequences with which they are operably-linked. In preferred embodiments, transcription of one of the FoxA2 genes is under the control of a promoter sequence (or other transcriptional regulatory sequence) that controls the expression of the recombinant gene in a cell-type in which expression is intended. It will also be understood that the recombinant gene can be under the control of transcriptional regulatory sequences which are the same or which are different from those sequences which control transcription of the naturally-occurring forms of FoxA2 polypeptide.

As used herein, the term “transfection” means the introduction of a nucleic acid, e.g., via an expression vector, into a recipient cell by nucleic acid-mediated gene transfer. Methods for transformation that are known in the art include any electrical, magnetic, physical, biological or chemical means. As used herein, “transfection” includes such specific techniques as electroporation, magnetoporation, Ca⁺⁺ treatment, injection, bombardment, retroviral infection and lipofection, among others. “Transformation”, as used herein, refers to a process in which a cell's genotype is changed as a result of the cellular uptake of exogenous DNA or RNA, and, for example, the transformed cell expresses a recombinant form of a FoxA2 polypeptide or, in the case of anti-sense expression from the transferred gene, the expression of a naturally-occurring form of the FoxA2 polypeptide is disrupted.

As used herein, the term “transgene” means a nucleic acid sequence (encoding, e.g., one of the FoxA2 polypeptides, or an antisense transcript thereto) that has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the FoxA2 polypeptides, e.g. either agonistic or antagonistic forms. However, transgenic animals in which the recombinant FoxA2 gene is silent are also contemplated, as for example, the FLP or CRE recombinase dependent constructs described below. Moreover, “transgenic animal” also includes those recombinant animals in which gene disruption of one or more FoxA2 genes is caused by human intervention, including both recombination and antisense techniques.

The term “treating” as used herein is intended to encompass curing as well as ameliorating at least one symptom of the condition or disease. “Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. Generally, “treatment” means the alleviation of the symptoms of an airway obstructive disease and/or preservation of lung function and/or the general improvement in the patient's perceived quality of life as regards the disease conditions and symptoms.

The term “upstream” identifies sequences proceeding in the opposite direction from expression; for example, the bacterial promoter is upstream from the transcription unit.

A “variant” as used herein, refers to an amino acid sequence that is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Similar minor variations may also include amino acid deletions or insertions, or both. Guidance in determining which amino acid residues may be substituted, inserted, or deleted without abolishing biological or immunological activity may be found using computer programs well known in the art, for example, DNASTAR software.

The term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops that, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

The present invention provides for both prophylactic and therapeutic methods of treating a subject at risk of (or susceptible to) a disorder or having a disorder associated with inflammation. Another aspect of the invention pertains to methods of modulating FoxA2 expression or activity for therapeutic purposes.

Accordingly, in an exemplary embodiment, the modulatory method of the invention involves contacting a cell with an active agent or compound that modulates one or more of the activities of FoxA2 activity associated with the cell.

An active compound that modulates FoxA2 activity can be an agent as described herein, such as a nucleic acid or a protein, a naturally-occurring target molecule of a FoxA2 protein (e.g., a FoxA2 ligand or substrate), a FoxA2 agonist or antagonist, a peptidomimetic of a FoxA2 agonist or antagonist, or other small molecule. In one embodiment, the active compound stimulates one or more FoxA2 activities. Examples of such stimulatory active compounds include active FoxA2 protein and a nucleic acid molecule encoding FoxA2 that has been introduced into the cell. In another embodiment, the active compound inhibits one or more FoxA2 activities. Examples of such inhibitory active compounds include antisense FoxA2 nucleic acid molecules, anti-FoxA2 antibodies, and FoxA2 inhibitors. These modulatory methods can be performed in vitro (e.g., by culturing the cell with the active compound) or, alternatively, in vivo (e.g., by administering the active compound to a subject). As such, the present invention provides methods of treating an individual afflicted with a disease or disorder characterized by aberrant or insufficient expression or activity of a FoxA2 protein or nucleic acid molecule such as a lung inflammation-related disorder. In one embodiment, the method involves administering an active compound (e.g., an active compound identified by a screening assay described herein), or combination of active compounds that modulates (e.g., upregulates or downregulates) FoxA2 expression or activity. In another embodiment, the method involves administering a chimeric FoxA2 protein or nucleic acid molecule as therapy to compensate for reduced, aberrant, or unwanted FoxA2 expression or activity.

The present invention also provides for replacement of FoxA2, whether by gene transfer vectors to express the normal allele or protein replacement with purified FoxA2 or recombinant FoxA2 or FoxA2 analogues, are beneficial for the treatment of pulmonary disorders.

The pathology of the lung disease includes idiopathic pulmonary fibrosis (IPF), desquamating interstitial pneumonitis (DIP), usual interstitial pneumonitis (UIP), non-specific interstitial pneumonitis (NSIP), and other forms of interstitial lung disease.

The active agent, e.g., the FoxA2 gene or protein, may be administered by aerosol or inhalation of a pharmaceutically useful preparation containing surfactant-like phospholipids, including phosphatidylglycerol, phosphatidylcholine.

The present invention provides for the use of a diagnostic screening tool based on the absence of FoxA2 in tissues or lavage lung material using immunohistochemistry, ELISA, Western blots, Mass spectroscopy, and protein sequencing.

The present invention provides methods to protect a subject from a respiratory disorder involving an airway obstructive disease such as asthma or chronic obstructive pulmonary disease.

The active agents useful in the present invention include factors or active agents that result in increased FoxA1/FoxA2 expression, which can be used as a therapeutic strategy to normalize lung epithelial cell differentiation associated with inflammatory or hereditary lung disease. Since FoxA2 controls goblet cell differentiation, its expression or agents that enhance or stabilize its expression or activity may also control epithelial cell function and differentiation. Furthermore, since FoxA1 and FoxA2 control a number of proteins involved in host defense, antioxidant surfactant homeostasis, and lung repair, active agents or methods to enhance or regulate their activities, are useful in the prevention or therapy of various inflammatory and hereditary lung diseases, including cystic fibrosis, asthma, emphysema, pulmonary fibrosis, bronchiectasis, and recurrent infection.

In one embodiment, drugs such as FK506 (an immunosuppressive agent, USAN tacrolimus; Prograf) are used to activate or regulate FoxA1/FoxA2 expression for therapy of various pulmonary disorders. In one embodiment, the active agent is at least one immunosuppressive agent selected from the group consisting of a nonsteroidal anti-inflammatory, a glucocorticoid, hydroxychloroquine, sulfaxalazine, methotrexate, aurothioglucose, aurothiomalate, auronofin, D-penicillamine, azathioprine, cyclophosphamide, cyclosporin A, FK506, and rapamycin. In another embodiment, the active agent is at least two immunosuppressive agent selected from the group consisting of a nonsteroidal anti-inflammatory, a glucocorticoid, hydroxychloroquine, sulfaxalazine, methotrexate, aurothioglucose, aurothiomalate, auronofin, D-penicillamine, azathioprine, cyclophosphamide, cyclosporin A, FK506, and rapamycin.

Alternatively, antisense or gene constructs, which inhibit or stimulate FoxA2 expression, are used as therapy for long-term disorders such as cystic fibrosis.

In one embodiment, provided are methods to protect a subject from an airway obstructive disease using gene therapy. Methods are provided for supplying FoxA2 function to cells of the lung and airway, such as smooth muscle and epithelial cells, by FoxA2 gene therapy. The FoxA2 gene, a modified FoxA2 gene, or a part of the gene may be introduced into the cell in a vector such that the gene remains extrachromosomal or may be integrated into the subjects chromosomal DNA for expression. These methods provide for administering to a subject in need of such treatment a therapeutically effective amount of a FoxA2 gene, or pharmaceutically acceptable composition thereof, for overexpressing the FoxA2 gene. Such methods of expressing the administered FoxA2 gene in the lungs and airway provide for: (1) preventing or alleving bronchial hyperresponsiveness; (2) preventing or alleving of an airway obstructive disease, e.g., bronchial hyperreactivity, airway hyperresponsiveness, asthma or chronic obstructive pulmonary disorder (“COPD”); (3) reducing the airway resistance response to inhaled natural or synthetic bronchoconstrictors or allergens or to exercise; and (4) enhancing responsiveness (relaxation) of airway tissues to β-agonists.

The FoxA2 gene or a part of the gene may or may not be integrated (covalently linked) to chromosomal DNA making up the genome of the subject's target cells. The genes may be introduced into the cell in a vector such that the gene remains extrachromosomal. In such a situation, the gene will be expressed by the cell from the extrachromosomal location. The cells may also be transformed where the exogenous DNA has become integrated into the chromosome so that it is inherited by daughter cells through chromosome replication. The gene may be introduced into an appropriate vector for extrachromosomal maintenance or for integration into the host. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of those in the art.

The polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence (SEQ ID NO:1 and SEQ ID NO:3) which encodes the mature polypeptide shown in FIG. 1, (SEQ ID NO:2) may be identical to the coding sequence or may be a different coding sequence which coding sequence, as a result of the redundancy or degeneracy of the genetic code, encodes the same mature polypeptide as the DNA of FIG. 1.

The polynucleotide which encodes for the mature FoxA2 polypeptide of FIG. 1 (SEQ ID NO:2) may include: only the coding sequence for the mature polypeptide; the coding sequence for the mature polypeptide and additional coding sequence; the coding sequence for the mature polypeptide (and optionally additional coding sequence) and non-coding sequence, such as introns or non-coding sequence 5′ and/or 3′ of the coding sequence for the mature polypeptide.

According to one embodiment of the present invention, a method is provided of supplying FoxA2 function to cells of the lung and airway, such as smooth muscle and epithelial cells, by FoxA2 gene therapy. The FoxA2 gene, a modified FoxA2 gene, or a part of the gene may be introduced into the cell in a vector such that the gene remains extrachromosomal. In such a situation, the FoxA2 gene will be expressed by the cell from the extrachromosomal location.

In accordance with the present invention, there is provided a method of treating airway obstructive disease comprising the administration to a patient in need of such treatment a therapeutically effective amount of a FoxA2 gene, or pharmaceutically acceptable composition thereof.

Such methods of providing the FoxA2 gene to the lungs and airway provide for: (a) preventing or alleving bronchial hyperresponsiveness; (b) preventing or alleving of an airway obstructive disease, e.g., bronchial hyperreactivity, airway hyperresponsiveness, asthma or chronic obstructive pulmonary disorder (“COPD”); (c) reducing the airway resistance response to inhaled natural or synthetic bronchoconstrictors or allergens or to exercise; and (d) enhancing responsiveness (relaxation) of airway tissues to β-agonists.

Therapeutic Administration

According to the method of treatment of the present invention, conditions subsumed under the above definition of airway obstructive disease, particularly asthma and chronic obstructive pulmonary disease (COPD) are treated in a patient in need of such treatment by administering to the patient a therapeutically effective amount of a compound of the invention, in such amounts and for such time as is necessary to achieve the desired result. By a “therapeutically effective amount” of a compound of the invention is meant a sufficient amount of the compound to effectively ameliorate the course of the disease and/or alleviate one or more symptoms of airway obstructive disease, or improve the quality of life in a patient at a reasonable benefit/risk ratio applicable to any medical treatment. It will be understood, however, that the total daily usage of the compounds and compositions of the present invention will be decided by the attending physician within the scope of sound medical judgment. The specific therapeutically effective dose level for any particular patient will depend upon a variety of factors including the severity of the disorder; the activity of the compound employed; the specific composition employed; the age, body weight, general health, sex and diet of the patient; the time of administration, route of administration, and rate of excretion of the compound employed; the duration of the treatment; drugs used in combination or coincidental with the specific compound employed; and like factors well known in the medical arts. Since some of these parameters vary from patient to patient, it is a well-known technique utilized by medical practitioners to determine the proper dose for a particular patient by “dose titrating” the patient; that is, by using the technique of starting with a dose lower than that required to obtain the desired effect, and gradually increasing the dose over time until the desired therapeutic benefit is obtained.

Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of those in the art.

The polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the mature polypeptide may be identical to the two coding variant sequences shown in FIG. 1, (SEQ ID NO:1 and SEQ ID NO:3) or may be a different coding sequence which coding sequence, as a result of the redundancy or degeneracy of the genetic code, encodes the same mature polypeptide as the DNA of FIG. 1.

The polynucleotide which encodes for the mature polypeptide of FIG. 2 (SEQ ID NO:1 and SEQ ID NO:3) may include: only the coding sequence for the mature polypeptide; the coding sequence for the mature polypeptide and additional coding sequence; the coding sequence for the mature polypeptide (and optionally additional coding sequence) and non-coding sequence, such as introns or non-coding sequence 5′ and/or 3′ of the coding sequence for the mature polypeptide.

The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

In vivo expression of FoxA2 transgenes is preferably by injection of transgenes directly into a specific tissue, such as direct intratracheal, intramuscular or intraarterial injection of naked DNA or of DNA-cationic liposome complexes, or to ex vivo transfection of host cells, with subsequent reinfusion.

Multiple approaches for introducing functional new genetic material into cells, both in vitro and in vivo are known. These approaches include integration of the gene to be expressed into modified retroviruses; integration into non-virus vectors; or delivery of a transgene linked to a heterologous promoter-enhancer element via liposomes; coupled to ligand-specification-based transport systems or the use of naked DNA expression vectors. Direct injection of transgenes into tissue produces localized expression PCT/US90/01515 (Felgner et al.) is directed to methods for delivering a gene coding for a pharmaceutical or immunogenic polypeptide to the interior of a cell of a vertebrate in vivo. PCT/US90/05993 (Brigham) is directed to a method for obtaining expression of a transgene in mammalian lung cells following either iv or intratracheal injection of an expression construct. While most gene therapy strategies have relied on transgene insertion into retroviral or DNA virus vectors, lipid carriers, may be used to transfect the lung cells of the host.

Preparation of Recombinant or Chemically Synthesized Nucleic Acids, vectors and Host Cells

Large amounts of the polynucleotides of the present invention may be produced by replication in a suitable host cell. Natural or synthetic polynucleotide fragments coding for a desired fragment will be incorporated into recombinant polynucleotide constructs, usually DNA constructs, capable of introduction into and replication in a prokaryotic or eukaryotic cell. Usually the polynucleotide constructs will be suitable for replication in a unicellular host, such as yeast or bacteria, but may also be intended for introduction to (with and without integration within the genome) cultured mammalian or plant or other eukaryotic cell lines.

The polynucleotides of the present invention may also be produced by chemical synthesis and may be performed on commercial, automated oligonucleotide synthesizers. A double-stranded fragment may be obtained from the single-stranded product of chemical synthesis either by synthesizing the complementary strand and annealing the strands together under appropriate conditions or by adding the complementary strand using DNA polymerase with an appropriate primer sequence.

Polynucleotide constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate, whether from a native FoxA2 protein or from other receptors or from secreted polypeptides of the same or related species, which allow the protein to cross and/or lodge in cell membranes, and thus attain its functional topology, or be secreted from the cell. Such vectors may be prepared by means of standard recombinant techniques well known in the art.

An appropriate promoter and other necessary vector sequences will be selected so as to be functional in the host, and may include, when appropriate, those naturally associated with FoxA2 genes. Many useful vectors are known in the art and may be obtained from such vendors as Stratagene, New England Biolabs, Promega Biotech, and others. Promoters such as the trp, lac and phage promoters, tRNA promoters and glycolytic enzyme promoters may be used in prokaryotic hosts. Useful yeast promoters include promoter regions for metallothionein, 3-phosphoglycerate kinase or other glycolytic enzymes such as enolase or glyceraldehyde-3-phosphate dehydrogenase, enzymes responsible for maltose and galactose utilization, and others. Appropriate non-native mammalian promoters might include the early and late promoters from SV40 or promoters derived from murine Moloney leukemia virus, mouse tumor virus, avian sarcoma viruses, adenovirus II, bovine papilloma virus or polyoma. In addition, the construct may be joined to an amplifiable gene so that multiple copies of the gene may be made.

In one embodiment, the nucleic acid construct will include at least one promoter selected from the group consisting of RNA polymerase III, RNA polymerase II, CMV promoter and enhancer, SV40 promoter, an HBV promoter, an HCV promoter, an HSV promoter, an HPV promoter, an EBV promoter, an HTLV promoter, an HIV promoter, and cdc25C promoter, a cyclin a promoter, a cdc2 promoter, a bmyb promoter, a DHFR promoter and an E2F-1 promoter.

Preferably, the nucleic acid construct will include at least one promoter selected from the group consisting of the promoter elements from the human FoxA1 or FoxA2 gene (SEQ ID NO:6 and SEQ ID NO:7).

Such vectors, which include a nucleic acid sequence(s) which encode for the FoxA2 protein, may also include at least one nucleic acid sequence encoding a therapeutic agent, whereby such vectors enable the expression of therapeutic agents in lung cells.

The vector hereinabove described may include a multiple cloning site to facilitate the insertion of DNA sequence(s) into the cloning vector. In general, the multiple cloning site includes “rare” restriction enzyme sites; i.e., sites which are found in eukaryotic genes at a frequency of from about one in every 10,000 to about one in every 100,000 base pairs. An appropriate vector in accordance with the present invention is thus formed by cutting the cloning vector by standard techniques at appropriate restriction sites in the multiple cloning site, and then ligating the DNA sequence encoding a therapeutic agent into the cloning vector.

The infectious viral particles then may be administered to a host, whereby the infectious viral particles will infect lung cells. The viral particles are administered in an amount effective to produce a therapeutic effect in a host. In one embodiment, the viral particles may be administered in an amount of from about 10⁶ to about 10¹² plaque forming units (pfu), preferably from about 10⁹ to about 10¹¹ pfu. The host may be a human or non-human animal host.

Therapeutic agents that may be encoded by a DNA or RNA sequence(s) placed in the vector include, any protein or polypeptide having a therapeutic effect. Such proteins or polypeptides include, but are not limited to, those encoded by DNA or RNA sequences encoding FoxA1, FoxA2 or lung surfactant proteins, such as SP-A, SP-B, SP-C, and SP-D for protection from lung injury; Clara Cell Secretory Protein (CCSP); the α-1-antitrypsin gene for treating lung fibrosis, cystic fibrosis, or emphysema; the cystic fibrosis transmembrane conductance regulator (CFTR); antioxidants such as, but not limited to, manganese superoxide dismutase (Mn—SOD), catalase, copper-zinc-superoxide dismutase (CuZn—SOD), extracellular superoxide dismutase (EC—SOD), and glutathione reductase, for treatment of acute lung injury, oxygen injury, or after chemical exposure to oxidants, infectious agents, shock, or for protection of the normal lung during chemotherapy for tumors (using bleomycin, adriamycin, or radiation); clotting factors, such as Factor VIII and Factor IX; and anti-tumor agents, such as, but not limited to, the Herpes Simplex thymidine kinase gene, wherein tumor killing is initiated by therapy with gancyclovir or acyclovir; GM-CSF (granulocyte-macrophage colony stimulating factor) which also may treat alveolar proteinosis, and cytokines such as TNF-α or Interleukin-1; and growth factors such as epidermal growth factor (EGF), and keratinocyte growth (KGF), for repair of or protection from injury after infection or oxygen therapy, bronchopulmonary dysplasia, or after therapy with lung oxidants such as antitumor agents, paraquot toxicity, or after exposure to toxins (e.g., alkylating agents, chemical warfare agents) or lung burns. In addition, the vector may include antisense DNA or RNA sequences.

Promoters may be a homologous or heterologous promoter. Such promoters include, but are not limited to, human globin promoters; viral thymidine kinase promoters, such as the Herpes Simplex thymidine kinase promoter; adenoviral late terminal repeats; retroviral LTRs; FoxA1 and Fox A2 promoters (SEQ ID NO:4, SEQ ID NO:5, SEQ ID NO:6, or SEQ ID NO:7); the Clara Cell secretory protein (CCSP) promoter; the β-actin promoter; and human growth hormone promoters. The promoter also may be the native promoter that controls the gene encoding the therapeutic agent. In general, the promoter will include a TATA box, transcription start signal, and a CAAT box or variation thereof.

For example, one may construct a vector in accordance with the present invention that includes the FoxA2 gene and a functional CFTR gene. The vector then may be administered to the respiratory epithelium in an effective therapeutic amount for the correction of the pulmonary deficit in patients with cystic fibrosis. In another example, vectors containing functional proteins may be delivered to the respiratory epithelium in order to correct deficiencies in such proteins. Such functional proteins include antioxidants, α-1-antitrypsin, CFTR, lung surfactant proteins, cytokines, and growth factors such as EGF and KGF, and may also include adenosine deaminase for treatment of severe combined immune deficiency, von Willebrand's factor for treatment of Christmas disease, and β-glucuronidase for treatment of Gaucher's disease. Also, vectors including genes encoding anti-cancer agents or anti-inflammatory agents may be administered to lung cells of a patient for the treatment of lung cancer or inflammatory lung disease.

While such expression vectors may replicate autonomously, they may also replicate by being inserted into the genome of the host cell, by methods well known in the art.

Expression and cloning vectors will likely contain a selectable marker, a gene encoding a protein necessary for survival or growth of a host cell transformed with the vector. The presence of this gene ensures growth of only those host cells that express the inserts. Typical selection genes encode proteins that a) confer resistance to antibiotics or other toxic substances, e.g. ampicillin, neomycin, methotrexate, etc.; b) complement auxotrophic deficiencies, or c) supply critical nutrients not available from complex media, e.g., the gene encoding D-alanine racemase for Bacilli. The choice of the proper selectable marker will depend on the host cell, and appropriate markers for different hosts are well known in the art.

The vectors containing the nucleic acids of interest can be transcribed in vitro, and the resulting RNA introduced into the host cell by well-known methods, e.g., by injection, or the vectors can be introduced directly into host cells by methods well known in the art, which vary depending on the type of cellular host, including electroporation; transfection employing calcium chloride, rubidium chloride, calcium phosphate, DEAE-dextran, or other substances; microprojectile bombardment; lipofection; infection (where the vector is an infectious agent, such as a retroviral genome); and other methods. The introduction of the polynucleotides into the host cell by any method known in the art, including, inter alia, those described above, will be referred to herein as “transformation.” The cells into which have been introduced nucleic acids described above are meant to also include the progeny of such cells.

Large quantities of the nucleic acids of the present invention may be prepared by expressing the FoxA2 nucleic acids or portions thereof in vectors or other expression vehicles in compatible prokaryotic or eukaryotic host cells. The most commonly used prokaryotic hosts are strains of Escherichia coli, although other prokaryotes, such as Bacillus subtilis or Pseudomonas may also be used.

Mammalian or other eukaryotic host cells, such as those of yeast, filamentous fungi, plant, insect, or amphibian or avian species, may also be useful for production of the proteins of the present invention. Propagation of mammalian cells in culture is per se well known. Examples of commonly used mammalian host cell lines are VERO and HeLa cells, Chinese hamster ovary (CHO) cells, and WI38, BHK, and COS cell lines, although it will be appreciated by the skilled practitioner that other cell lines may be appropriate, e.g., to provide higher expression, desirable glycosylation patterns, or other features.

Clones are selected by using markers depending on the mode of the vector construction. The marker may be on the same or a different DNA molecule, preferably the same DNA molecule. In prokaryotic hosts, the transformant may be selected, e.g., by resistance to ampicillin, tetracycline or other antibiotics. Production of a particular product based on temperature sensitivity may also serve as an appropriate marker.

The methods of the present invention utilize gene sequences encoding a peptide containing at least 100 or more amino acids with at least 70% sequence identity to an amino acid sequence of FoxA2. In preferred embodiments, the methods of the present invention utilize gene sequences encoding a peptide containing at least 200 or more amino acids with at least 70% sequence identity to an amino acid sequence of FoxA2. In more preferred embodiments, the methods of the present invention utilize gene sequences encoding a peptide containing at least 300 or more amino acids with at least 80% sequence identity to an amino acid sequence of FoxA2 and the amino acid sequence of FoxA2 is the amino acid sequence of peptide of Sequence No. 2 from about position 34 to about position 341 with at least 85% sequence identity to the amino acid sequence of FoxA2 is also within the invention. Preferably, the peptide shares 100% sequence identity with the amino acid sequence of FoxA2.

“Identity”, as used herein, refers to the subunit sequence similarity between two polymeric molecules, e.g., two peptide molecules. When a subunit position in both of the two molecules is occupied by the same monomeric subunit, e.g., if a position in each of two peptides is occupied by serine, then they are identical at that position. The identity between two sequences is a direct function of the number of matching or identical positions, e.g., if half (e.g., 5 positions in a polymer 10 subunits in length), of the positions in two peptide or compound sequences are identical, then the two sequences are 50% identical; if 90% of the positions, e.g., 9 of 10, are matched, the two sequences share 90% sequence identity. By way of example, the amino acid sequences VRGLQP and HAFLQP share 50% sequence identity.

Gene Therapy

According to the present invention, a method is provided of supplying FoxA2 function to airway cells of an appropriate subject. The wild-type FoxA2 gene or a part of the gene may be introduced into the cell in a vector such that the gene remains extrachromosomal. In such a situation, the gene will be expressed by the cell from the extrachromosomal location. Vectors for introduction of genes both for recombination and for extrachromosomal maintenance are known in the art, and any suitable vector may be used. Methods for introducing DNA into cells such as electroporation, calcium phosphate co-precipitation and viral transduction are known in the art, and the choice of method is within the competence of the routineer.

Gene therapy is carried out according to generally accepted methods. Generally, a virus or plasmid vector, containing a copy of the FoxA2 gene linked to expression control elements and capable of replicating inside the airway cells, is prepared. Suitable vectors are known, such as disclosed in U.S. Pat. No. 5,252,479, incorporated herein in its entirety by reference. The vector is then introduced into the patient, either locally at the site of the airway tissue or systemically. If the transfected gene is not permanently incorporated into the genome of the targeted cells, the treatment may have to be repeated periodically.

Gene transfer systems known in the art may be useful in the practice of the gene therapy methods of the present invention. These include viral and nonviral transfer methods. A number of viruses have been used as gene transfer vectors, including papovaviruses, e.g., SV40, adenovirus, vaccinia virus, adeno-associated virus, herpesviruses including HSV and EBV, and retroviruses of avian, murine and human origin. Most human gene therapy protocols have been based on disabled murine retroviruses.

Nonviral gene transfer methods known in the art include chemical techniques such as calcium phosphate co-precipitation; mechanical techniques, for example microinjection; membrane fusion-mediated transfer via liposomes; and direct DNA uptake and receptor-mediated DNA transfer. Viral-mediated gene transfer can be combined with direct in vivo gene transfer using liposome delivery. Alternatively, the retroviral vector producer cell line can be injected into airway tissues. Injection of producer cells would then provide a continuous source of vector particles.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged.

Liposome/DNA complexes have been shown to be capable of mediating direct in vivo gene transfer. While in standard liposome preparations the gene transfer process is nonspecific, localized in vivo uptake and expression have been reported in tumor deposits, for example, following direct in situ administration.

Gene transfer techniques that target DNA directly to lung tissues, e.g., airway smooth muscle and epithelial cells, is preferred. Receptor-mediated gene transfer, for example, is accomplished by the conjugation of DNA (usually in the form of covalently closed supercoiled plasmid) to a protein ligand via polylysine. Ligands are chosen on the basis of the presence of the corresponding ligand receptors on the cell surface of the target cell/tissue type. These ligand-DNA conjugates can be injected directly into the blood if desired and are directed to the target tissue where receptor binding and internalization of the DNA-protein complex occurs. To overcome the problem of intracellular destruction of DNA, co-infection with adenovirus can be included to disrupt endosome function.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). Preferably, when the subject is a human, a vector such as the gibbon ape leukemia virus (GaLV) is utilized. A number of additional retroviral vectors can incorporate multiple genes. All of these vectors can transfer or incorporate a gene for a selectable marker so that transduced cells can be identified and generated. By inserting a FoxA2 gene sequence (including promoter region) of interest into the viral vector, along with another gene that encodes the ligand for a receptor on a specific target cell, for example, the vector is now target specific. Retroviral vectors can be made target specific by inserting, for example, a polynucleotide encoding a sugar, a glycolipid, or a protein. Preferred targeting is accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector containing the FoxA2 polynucleotide.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence that enables the packaging mechanism to recognize an RNA transcript for encapsidation. Helper cell lines that have deletions of the packaging signal include but are not limited to PSI 2, PA317 and PA12, for example. These cell lines produce empty virions, since no genome is packaged. If a retroviral vector is introduced into such cells in which the packaging signal is intact, but the structural genes are replaced by other genes of interest, the vector can be packaged and vector virion produced.

Another targeted delivery system for FoxA2 polynucleotide is a colloidal dispersion system. Colloidal dispersion systems include macromolecule complexes, nanocapsules, microspheres, beads, and lipid-based systems including oil-in-water emulsions, micelles, mixed micelles, and liposomes. The preferred colloidal system of this invention is a liposome. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of aqueous buffer containing large macromolecules. RNA, DNA and intact virions can be encapsulated within the aqueous interior and be delivered to cells in a biologically active form. In addition to mammalian cells, liposomes have been used for delivery of polynucleotides in plant, yeast and bacterial cells. In order for a liposome to be an efficient gene transfer vehicle, the following characteristics should be present: (1) encapsulation of the genes of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information.

The constructs for use in the invention include several forms, depending upon the intended use of the construct. Thus, the constructs include vector, transcriptioned cassettes, expression cassettes and plasmids. The transcriptional and translational initiation region (also sometimes referred to as a “promoter,”), preferably comprises a transcriptional initiation regulatory region and a translational initiation regulatory region of untranslated 5′ sequences, “ribosome binding sites,” responsible for binding mRNA to ribosomes and translational initiation. It is preferred that all of the transcriptional and translational functional elements of the initiation control region are derived from or obtainable from the same gene. In some embodiments, the promoter will be modified by the addition of sequences, such as enhancers, or deletions of nonessential and/or undesired sequences. By “obtainable” is intended a promoter having a DNA sequence sufficiently similar to that of a native promoter to provide for the desired specificity of transcription of a DNA sequence of interest. It includes natural and synthetic sequences as well as sequences which may be a combination of synthetic and natural sequences.

The nucleic acid constructs generally will be provided as transcriptional cassettes. An intron optionally may be included in the construct, preferably >/=100 bp and placed 5′ to the coding sequence. Generally it is preferred that the construct not become integrated into the host cell genome and the construct is introduced into the host as part of a non-integrating expression cassette. A coding sequence is “operably linked to” or “under the control of” transcriptional regulatory regions in a cell when DNA polymerase will bind the promoter sequence and transcribe the coding sequence into mRNA, either a sense strand or an antisense strand. Thus, the nucleic acid sequence includes DNA sequences that encode polypeptides have the biological activity of FoxA2 that are directly or indirectly responsible for a therapeutic effect, as well as nucleotide sequences coding for nucleotide sequences such as antisense sequences and ribozymes.

In one embodiment, constructs are used that produce long-term effects in vivo, either by integration into host cell genomic DNA at high levels or by persistence of the transcription cassette in the nucleus of cells in vivo in stable, episomal form. Integration of the transcription cassette into genomic DNA of host cells in vivo is facilitated by administering the transgene in a linearized form (either in the coding region alone, or the coding region together with 5′ and 3′ regulatory sequences, but without any plasmid sequences present).

The constructs for use in the invention include several forms, depending upon the intended use of the construct. Thus, the constructs include vectors, transcriptional cassettes, expression cassettes and plasmids. The transcriptional and translational initiation region (also sometimes referred to as a “promoter,”), preferably comprise a transcriptional initiation regulatory region and a translational initiation regulatory region of untranslated 5′ sequences, “ribosome binding sites,” responsible for binding mRNA to ribosomes and translational initiation. It is preferred that all of the transcriptional and translational functional elements of the initiation control region are derived from or obtainable from the same gene. In some embodiments, the promoter will be modified by the addition of sequences, such as enhancers, or deletions of nonessential and/or undesired sequences. By “obtainable” is intended a promoter having a DNA sequence sufficiently similar to that of a native promoter to provide for the desired specificity of transcription of a DNA sequence of interest. It includes natural and synthetic sequences as well as sequences which may be a combination of synthetic and natural sequences.

For the transcriptional initiation region, or promoter element, any region may be used with the proviso that it provides the desired level of transcription of the DNA sequence of interest. The transcriptional initiation region may be native to or homologous to the host cell, and/or to the DNA sequence to be transcribed, or foreign or heterologous to the host cell and/or the DNA sequence to be transcribed. By foreign to the host cell is intended that the transcriptional initiation region is not found in the host into which the construct comprising the transcriptional initiation region is to be inserted. By foreign to the DNA sequence is intended a transcriptional initiation region that is not normally associated with the DNA sequence of interest. Efficient promoter elements for transcription initiation include the SV40 (simian virus 40) early promoter, the RSV (Rous sarcoma virus) promoter, the Adenovirus major late promoter, and the human CMV (cytomegalovirus) immediate early 1 promoter.

Inducible promoters also find use with the subject invention where it is desired to control the timing of transcription. Examples of promoters include those obtained from a beta-interferon gene, a heat shock gene, a metallothionein gene or those obtained from steroid hormone-responsive genes, including insect genes such as that encoding the ecdysone receptor. Such inducible promoters can be used to regulate transcription of the transgene by the use of external stimuli such as interferon or glucocorticoids. Since the arrangement of eukaryotic promoter elements is highly flexible, combinations of constitutive and inducible elements also can be used. Tandem arrays of two or more inducible promoter elements may increase the level of induction above baseline levels of transcription which can be achieved when compared to the level of induction above baseline which can be achieved with a single inducible element.

Generally, the regulatory sequence comprises DNA up to about 1.5 Kb 5′ of the transcriptional start of a gene, but can be significantly smaller. This regulatory sequence may be modified at the position corresponding to the first codon of the desired protein by site-directed mutagenesis or by introduction of a convenient linker oligonucleotide by ligation, if a suitable restriction site is found near the N-terminal codon. In the ideal embodiment, a coding sequence with a compatible restriction site may be ligated at the position corresponding to codon #1 of the gene. This substitution may be inserted in such a way that it completely replaces the native coding sequence and thus the substituted sequence is flanked at its 3′ end by the gene terminator and polyadenylation signal.

Transcriptional enhancer elements optionally may be included in the expression cassette. By transcriptional enhancer elements is intended DNA sequences which are primary regulators of transcriptional activity and which can act to increase transcription from a promoter element, and generally do not have to be in the 5′ orientation with respect to the promoter in order to enhance transcriptional activity. The combination of promoter and enhancer element(s) used in a particular expression cassette can be selected by one skilled in the art to maximize specific effects. Different enhancer elements can be used to produce a desired level of transgene expression in a wide variety of tissue and cell types. For example, the human CMV immediate early promoter-enhancer element can be used to produce high-level transgene expression in many different tissues in vivo.

Examples of other enhancer elements that confer a high level of transcription on linked genes in a number of different cell types from many species include enhancers from SV40 and RSV-LTR. The SV40 and RSV-LTR are essentially constitutive. They may be combined with other enhancers that have specific effects, or the specific enhancers may be used alone. Thus, where specific control of transcription is desired, efficient enhancer elements that are active only in a tissue-, developmental-, or cell-specific fashion include immunoglobulin, interleukin-2 (IL-2) and beta-globin enhancers are of interest. Tissue-, developmental-, or cell-specific enhancers can be used to obtain transgene expression in particular cell types, such as B-lymphocytes and T-lymphocytes, as well as myeloid, or erythroid progenitor cells. Alternatively, a tissue-specific promoter such as that derived from the FoxA2 gene can be fused to a very active, heterologous enhancer element, such as the SV40 enhancer, in order to confer both a high level of transcription and tissue-specific transgene transcription. In addition, the use of tissue-specific promoters, such as LCK, may allow targeting of transgene transcription to T lymphocytes. Tissue specific transcription of the transgene may be important, particularly in cases where the results of transcription of the transgene in tissues other than the target tissue would be deleterious.

Tandem repeats of two or more enhancer elements or combinations of enhancer elements may significantly increase transgene expression when compared to the use of a single copy of an enhancer element; hence enhancer elements find use in the expression cassette. The use of two different enhancer elements from the same or different sources flanking or within a single promoter can in some cases produce transgene expression in each tissue in which each individual enhancer acting alone would have an effect, thereby increasing the number of tissues in which transcription is obtained. In other cases, the presence of two different enhancer elements results in silencing of the enhancer effects. Evaluation of particular combinations of enhancer elements for a particular desired effect or tissue of expression is within the level of skill in the art.

Although generally it is not necessary to include an intron in the expression cassette, an intron comprising a 5′ spice site (donor site) and a 3′ spice site (acceptor site) separated by a sufficient intervening sequence to produce high level, extended in vivo expression of a transgene administered iv or ip can optionally be included. Generally, an intervening sequence of about 100 bp produces the desired expression pattern and/or level, but the size of the sequence can be varied as need to achieve a desired result. The optional intron placed 5′ to the coding sequence results in high level extended in vivo expression of a transgene administered iv or ip but generally is not necessary to obtain expression. Optimally, the 5′ intron specifically lacks cryptic splice sites that result in aberrantly spliced mRNA sequences.

Alternatively, the intervening sequence may be placed 3′ to the translational stop codon and the transcriptional terminator or inside the coding region. The intron can be a hybrid intron with an intervening sequence or an intron taken from a genomic coding sequence. An intron 3′ to the coding region, a 5′ intron which is of less than 100 bp, or an intron which contains cryptic splice sites may under certain condition substantially reduce the level of transgene expression produced in vivo. However, unexpectedly, a high level of in vivo expression of a transgene can be achieved using a vector that lacks an intron. Such vectors therefore are of particular interest for in vivo transfection.

Downstream from and under control of the transcriptional initiation regulatory regions is a multiple cloning site for insertion of a nucleic acid sequence of interest that will provide for one or more alterations of host genotype and modulation of host phenotype. Conveniently, the multiple cloning site may be employed for a variety of nucleic acid sequences in an efficient manner. The nucleic acid sequence inserted in the cloning site may have any open reading frame encoding a polypeptide of interest, for example, an enzyme, with the proviso that where the coding sequence encodes a polypeptide of interest, it should lack cryptic splice sites which can block production of appropriate mRNA molecules and/or produce aberrantly spliced or abnormal mRNA molecule. The nucleic acid sequence may be DNA; it also may be a sequence complementary to a genomic sequence, where the genomic sequence may be one or more of an open reading frame, an intron, a non-coding leader sequence, or any other sequence where the complementary sequence will inhibit transcription, messenger RNA processing, for example splicing, or translation.

The incidence of integration of the transcription cassette into genomic DNA may be increased by incorporating a purified retroviral enzyme, such as the HIV-1 integrase enzyme, into the lipid carrier-DNA complex. Appropriate flanking sequences are placed at the 5′ and 3′ ends of the nucleic acid. These flanking sequences have been shown to mediate integration of the HIV-1 DNA into host cell genomic DNA in the presence of HIV-1 integrase. Alternatively, the duration of the expression of the exogenous nucleic acid in vivo can be prolonged by the use of constructs that contain non-transforming sequences of a virus such as Epstein-Barr virus, and sequences such as oriP and EBNA-1 which appear to be sufficient to allow heterologous DNA to be replicated as an episome in mammalian cells.

The termination region employed primarily will be one of convenience, since termination regions appear to be relatively interchangeable. The termination region may be native to the intended nucleic acid sequence of interest, or may be derived from another source. Convenient termination regions are available and include the 3′ end of a gene terminator and polyadenylation signal from the same gene from which the 5′ regulatory region is obtained. Adenylation residues, preferably more than 32 and up to 200 or more as necessary may be included in order to stabilize the mRNA. Alternatively, a terminator and polyadenylation signal from different gene/genes may be employed with similar results. Specific sequences which regulate post-transcriptional mRNA stability may optionally be included. For example, certain polyA sequences and beta-globin mRNA elements can increase mRNA stability, whereas certain AU-rich sequences in mRNA can decrease mRNA stability. In addition, AU regions in 3′ non-coding regions may be used to destabilize mRNA if a short half-life mRNA is desirable for the gene of interest.

Aerosol Administration

The mammalian host may be any mammal. Thus, the subject application finds use in domestic animals, e.g., equine, feed stock, such as bovine, ovine, and porcine, as well as primates, particularly humans. In the method of the invention, transformation in vivo is obtained by introducing a non-integrating therapeutic plasmid into the mammalian host, preferably complexed to a lipid carrier, particularly a cationic lipid carrier more particularly, for human use or for repeated applications a biodegradable lipid carrier. For introduction into the mammalian host any physiologically acceptable medium may be employed for administering the DNA or lipid carriers, such as deionized water, 5% dextrose in water, and the like. Other components may be included in the formulation such as stabilizers, biocides, etc, providing that they meet the criteria outlined above, i.e. do not cause aggregation of the complexes. The various components listed above find extensive exemplification in the literature and need not be described in particular here.

For aerosol delivery in humans or other primates, the aerosol is generated by a medical nebulizer system that delivers the aerosol through a mouthpiece, facemask, etc. from which the mammalian host can draw the aerosol into the lungs. Various nebulizers are known in the art and can be used in the method of the present invention. The selection of a nebulizer system depends on whether alveolar or airway delivery (i.e., trachea, primary, secondary or tertiary bronchi, etc.), is desired. The particular nucleic acid composition is chosen that is not too irritating at the required dosage.

Nebulizers useful for airway delivery include those typically used in the treatment of asthma. Such nebulizers are also commercially available. The amount of compound used will be an amount sufficient to provide for adequate transfection of cells after entry of the DNA or complexes into the lung and airway and to provide for a therapeutic level of transcription and/or translation in transfected cells. A therapeutic level of transcription and/or translation is a sufficient amount to prevent, treat, or palliate a disease of the host mammal following administration of the nucleic acid composition to the host mammal's lung, particularly the alveoli or bronchopulmonary and bronchiolopulmonary smooth muscle and epithelial cells of the trachea, bronchi, bronchia, bronchioli, and alveoli. Thus, an effective amount of the aerosolized nucleic acid preparation, is a dose sufficient to effect treatment, that is, to cause alleviation or reduction of symptoms, to inhibit the worsening of symptoms, to prevent the onset of symptoms, and the like. The dosages of the preset compositions that constitute an effective amount can be determined in view of this disclosure by one of ordinary skill in the art by running routine trials with appropriate controls. Comparison of the appropriate treatment groups to the controls will indicate whether a particular dosage is effective in preventing or reducing particular symptoms.

The total amount of nucleic acid delivered to a mammalian host will depend upon many factors, including the total amount aerosolized, the type of nebulizer, the particle size, breathing patterns of the mammalian host, severity of lung disease, concentration of the nucleic acid composition in the aerosolized solution, and length of inhalation therapy. Thus, the amount of expressed protein measured in the airways may be substantially less than what would be expected to be expressed from the amount of nucleic acid present in the aerosol, since a large portion of the complex may be exhaled by the subject or trapped on the interior surfaces of the nebulizer apparatus. For example, approximately one third of the nucleic acid composition dose that is placed into the nebulizer remains in the nebulizer and associated tubing after inhalation is completed. This is true regardless of the dose size, duration of inhalation, and type of nebulizer used. Moreover, resuspension of the residue and readministration does not significantly increase the dose delivered to the subject; about one third remains in the nebulizer. Additionally, efficiency of expression of the encoded protein will vary widely with the expression system used.

Despite the interacting factors described above, one of ordinary skill in the art will be able readily to design effective protocols, particularly if the particle size of the aerosol is optimized. Based on estimates of nebulizer efficiency, an effective dose delivered usually lies in the range of about 1 mg/treatment to about 500 mg/treatment, although more or less may be found to be effective depending on the subject and desired result. It is generally desirable to administer higher doses when treating more severe conditions. Generally, the nucleic acid is not integrated into the host cell genome, thus if necessary, the treatment can be repeated on an ad hoc basis depending upon the results achieved. If the treatment is repeated, the mammalian host is monitored to ensure that there is no adverse immune response to the treatment. The frequency of treatments depends upon a number of factors, such as the amount of nucleic acid composition administered per dose, as well as the health and history of the subject.

Experimental Procedures

Materials and Methods

Animals and Transgene Genotype. FoxA2loxP/loxP mice are generated at the University of Pennsylvania (Sund et al., 2000) maintained as homozygous. Homologous recombination between loxP sites is accomplished utilizing the (tetO)7CMV-Cre mice (Sauer, 1998), kindly provided by Dr. Corrinne Lobe, University of Toronto. For lung-specific, doxycycline-induced recombination, the FoxA2-rtTA-/tg or CCSP-rtTA-/tg transgenic lines are used (Perl et al., 2002a; Tichelaar et al., 2000). Triple transgenic mice, termed FoxA2Δ/Δ, are generated by crossing (tetO)7-Cre-/tg/FoxA2loxP/loxP and SPC-rtTA-/tg/FoxA2loxP/loxP or CCSP-rtTA-/tg/FoxA2loxP/loxP. FoxA2loxP/loxP littermates lacking either rtTA or Cre alleles serve as controls. Transgenic mice are identified by PCR using genomic DNA from the tails of fetal and postnatal mice, as previously described (Perl et al., 2002a).

Animal Husbandry and Doxycycline Administration. Animals are maintained in pathogen-free conditions according to protocols approved by Institutional Animal Care and Use Committee at Cincinnati Children's Hospital Research Foundation. All animals are housed in humidity- and temperature-controlled rooms on a 12:12-hour light dark cycle and are allowed food and water ad libitum. There is no serologic evidence of pulmonary pathogens or bacterial infections in sentinel mice maintained within the colony. No serological evidence of viral infection or histological evidence of bacterial infection is detected in representative mice. Gestation is dated by detection of the vaginal plug. Dams bearing double- and triple-transgenic pups are maintained on doxycycline in food (25 mg/g; Harlan Teklad, Madison, Wis.) for various time spans. The mice are killed by injection of anesthetic and exsanguinated.

Mouse Models with Goblet Cell Hyperplasia. Lung samples from each of the following mice and controls are fixed with 4% paraformaldehyde and embedded in paraffin for Alcian blue and FoxA2 staining. Ovalbumin challenge model: BALB/c mice are obtained from the National Cancer Institute (Frederick, Md.) and housed under specific pathogen-free conditions. Mice are treated twice by intraperitoneal injection with 100 μg ovalbumin (OVA, Sigma, grade V) and 1 mg aluminum hydroxide (alum) followed by two 50 μg OVA or saline intranasal treatments 3 days apart, starting at least 10 days after the second sensitization, as previously described (Mishra et al., 2001). Mice are killed 18 hours after the second intranasal administration. Conditional expression of IL-13: Four week old bitransgenic mice bearing CC10-rtTA and (tetO)7CMV-IL-13 transgenes, identified by Southern blot analysis, are fed doxycycline in the food for two weeks, inducing expression of IL-13 in the lung. Histological analysis of the lung from double transgenic mice revealed marked perivascular and peribronchial inflammatory lesions, thickened basement membranes, smooth muscle hyperplasia, deposition of collagen, and production of mucus.

IL-4 treatment of Stat-6−/− mice: Signal transducer and activator of transcription-6-deficient (Stat-6−/−) mice on a BALB/c background are originally obtained from Michael Grusby (Harvard, Cambridge, Mass.) and are bred at the University of Cincinnati College of Medicine. Briefly, control and Stat-6−/− adult mice are treated daily with 2 μg IL-4 (in 40 μl) or 40 μl normal saline intratracheally for 4 days. Mice are killed one day after the last intratracheal inoculation and the tissues stained for FoxA2 with eosin as counterstain.

IL-4 overexpression mouse model: The generation of IL-4 expressing mice is described previously using the CCSP promoter that is selective for conducting airway epithelial cells (Rankin et al., 1996). Sections from adult CCSP-IL-4 mice and control (n=4 per group), are prepared for Alcian blue and FOXA1 staining.

FoxA2 deficient mouse model: Adult Sftpc−/− mice (129/Sv) strain spontaneously develop goblet cell hyperplasia and enhanced MUC5A/C staining in the conducting airways (Glasser et al., 2003). Adult Sftpc−/− mice and littermate controls are prepared for Alcian blue, FoxA2 double staining, n=4 per group.

Histology and immunohistochemistry. Tissues from fetal and neonatal lungs are prepared as previously described (Wert et al., 2000). Antibodies used are generated to: pro-FoxA2 (1:1000, rabbit polyclonal, AB3428, Chemicon), CCSP (1:7500, rabbit polyclonal, kindly provided by Dr, Barry Stripp, University of Pittsburgh), SP-B (1:1000, rabbit polyclonal, generated in this lab), TTF-1 (1:1000, rabbit polyclonal, kindly provided by Dr. Roberto DiLauro), platelet endothelial cell adhesion molecule-1 (PECAM-1) (1:500, rat polyclonal, clone CD31, Pharmingen), FoxA2 (1:800, sheep immunoaffinity purified IgG, Upstate Biotechnology), MUC5A/C (1:500, chicken polyclonal antibody, kindly provided by Dr. Samuel Ho), phosphohistone H3 (1:100, rabbit polyclonal, United States Biological). Immunostaining is performed as described previously (Zhou et al., 1996) using a FoxA1 (1:50, monoclonal anti-mouse antibody generated in this laboratory) using a Mom-kit (Vector Laboratories, Inc.). After staining for FoxA2, lung sections are then counterstained for neutral or acidic mucins cells with periodic acid Schiff (PAS) reaction or Alcian blue PH2.5 method (Poly Scientific R&D Corp.). Elastin staining is performed using orcein as directed by the manufacturer (Poly Scientific R&D Corp.). All experiments shown are representative of findings from at least two independent dams, generating at least four triple transgenic offspring that are compared with littermates.

Lung Morphometry. Morphometric measurements are performed on inflation-fixed lungs on postnatal day 16, n=3-5 for each genotype. At least 5 representative fields are studied in each mouse. Slides are viewed by using a 20× objective, and the images transferred by video camera to computer screen using METAMORPH imaging software (Universal Imaging, West Chester, Pa.). Percent fractional airspace areas and percent fractional areas of lung parenchyma are determined as previously described (Liu et al., 2003; Wert et al., 2000). Percent fractional area of respiratory airspace is determined by airspace surface area divided by total area. Pairwise t-test is used to determine significant changes at p<0.05.

RNA analysis. S1 nuclease protection assay and RNAse protection assay are performed as described previously (Jobe et al., 2000; Rausa et al, 2000). SP-A, SP-B, FoxA2, CCSP, and FoxA2 mRNA are quantified by S1 nuclease protection assay or RNAse protection assay with ribosomal protein L32 as an internal control.

Protein measurements. Mice are anesthetized, exsanguinated, and BALF collected as described previously (Wert et al., 2000). IL-13, IL-4, IFN-γ, IL-5, GM-CSF, MIP-2 and KC are measured in the supernatant of lung homogenates using ELISA Kits (R&D Systems, Minneapolis, Minn.) according to the manufacture's protocol. Western blot analysis for SP-B and FoxA2 are performed on lung homogenates from FoxA2Δ/Δ and control littermates at PN16 as previously described (Melton et al., 2003).

Pulmonary function studies. Lung mechanics are assessed in adult CCSP-rtTA, FoxA2Δ/Δ and control mice at 7 weeks of ages by a computerized Flexi Vent system (SCIREQ, Montreal, PQ, Canada), as previously described (Liu et al., 2003; Schuessler and Bates 1995).

Transcription of the MUC5A/C promoter in vitro. The MUC5A/C promoter-luciferase construct, consisting of 3.7 kb of the mouse gene is kindly provided by Dr. Carol Basbaum, University of San Francisco (Li et al., 1998). The construct is transfected in H292 cells, a pulmonary cell line that produces MUC5A/C in vitro. Fugene 6 (Roche Molecular, Biochemicals, Indianapolis, Ind.) is used for transfection in accordance with the manufacturer's directions. Trans-retinoic acid, 3 ng/ml, is added to H292 cells 24 hours after transfection for a positive control. Forty-eight hours after transfection, luciferase activity is assessed and normalized for co-transfection by β-galactosidase activity. All transfections are performed in triplicate, mean±s.e.m.

Statistical analysis. ANOVA or Student's T-test are used to determine the levels of difference between groups, P values for significance are set to 0.05. Values for all measurements are expressed as the mean±s.e.m.

Human tissues. Human lung tissue is obtained at autopsy or lobectomy under protocols approved by the Committee on Human Research, Vanderbilt University.

Results

Conditional deletion of FoxA2 in the lung. Triple transgenic FoxA2loxP/loxP, FoxA2-rtTA-/tg, (tetO)7-Cre-/tg (FoxA2-rtTA compound mice) and FoxA2loxP/loxP, CCSP-rtTA-/tg, (tetO)7-Cre-/tg (CCSP-rtTA compound mice) mice are produced in which FoxA2 is selectively deleted in subsets of respiratory epithelial cells in the developing lung. In these mice, the rtTA (reverse tetracycline responsive transactivator) is expressed in lung epithelial cells under control of FoxA2 or CCSP promoter elements. In the presence of doxycycline, rtTA binds to the (tetO)7CMV promoter, activating expression of Cre-recombinase, deleting exon 3 of the FoxA2 gene (FIG. 1A). When FoxA2-rtTA compound and CCSP-rtTA compound mice are maintained on doxycycline from E0, pups are born at the expected Mendelian frequency. At birth, body and lung weights are not different in triple transgenic mice and their controls.

Immunohistochemistry demonstrates deletion of FoxA2. To assess the efficiency of Cre-mediated gene deletion in FoxA2-rtTA compound and CCSP-rtTA compound mice, dams are maintained on doxycycline from E0. FoxA2 staining is assessed at postnatal day 16 (PN16). In wild type mice, FoxA2 is detected in epithelial cells of both conducting airways and in alveolar epithelial type II cells, consistent with previous studies (Zhou et al., 1996). FoxA2 staining is absent in most epithelial cells of peripheral conducting airways and alveoli in FoxA2-rtTA compound mice (FIG. 1B). Under control of CCSP-rtTA, FoxA2 is deleted primarily in conducting airways and in restricted subsets of peripheral respiratory epithelial cells. The extent of deletion of FoxA2 is variable in both FoxA2 and CCSP-rtTA compound mice, ranging from complete absence of FoxA2 staining to heterogeneous persistence of staining. FoxA2 deletion is assessed by RNAase protection analysis (FIG. 1C), demonstrating the variable, but marked decrease in FoxA2 mRNA in the lungs of FoxA2Δ/Δ mice.

Effects of FoxA2 deletion on lung morphogenesis. When FoxA2-rtTA compound mice are maintained on doxycycline from E0, approximately 50% of the pups died between PN1 and PN30 (n=24 litters). At E16.5-18.5, lung morphology is not perturbed in FoxA2Δ/Δ pups (FIG. 2A,B). However, by PN3, fewer peripheral lung saccules and decreased alveolar septation are observed, indicating an abnormality in postnatal alveolarization (FIG. 2C,D). Airspace enlargement, focal neutrophilic infiltrations and goblet cell hyperplasia are observed at PN16 and thereafter (FIG. 2E,F). Morphometric analysis of fractional airspace and fractional respiratory parenchyma supported the histologic assessment of the alveolar abnormalities in FoxA2 but not CCSP-rtTA deleted mice (FIG. 3). Increased numbers of neutrophils and macrophages are observed in bronchoalveolar lavage fluid of one month old mice after deletion of FoxA2 (supplemental FIG. 1). Differential cell counts showed a significant increase in neutrophils (10±4.2%) compared to littermate controls ((0.25±0.5%), mean±s.e.m. by ANOVA, P<0.05). Some neutrophils stained for Ly-6 and the alveolar macrophages are generally MAC-3 positive (data not shown). Repeated bacterial cultures of the lung indicated no pulmonary infection. Likewise, sentinel mice did not indicate bacteria or viral pathogens in the colony. No bacteria are found on lung sections or are observed on cytospins of BALF (data not shown).

Goblet cell hyperplasia after deletion of FoxA2. Goblet cell hyperplasia is observed in bronchi and bronchioles after deletion of FoxA2 in both FoxA2-rtTA and CCSP-rtTA compound mice (FIG. 1B, inserts). In the control mouse lung, goblet cells indicated by Alcian blue or MUC5A/C staining are rarely observed. In contrast, staining for acidic and neutral mucins is observed in conducting airways of FoxA2Δ/Δ mice as assessed by Alcian blue and periodic acid Schiff staining respectively (FIG. 4C-F). Likewise, extensive MUC5A/C staining is detected at the sites of goblet cell hyperplasia (FIG. 4G,H). CCSP-rtTA compound mice maintained on doxycycline from E0 survived postnatally. Airspace enlargement and neutrophilic infiltrates are not detected in the peripheral lung of CCSP-rtTA triple transgenic mice. Loss of FoxA2 staining is less extensive in alveolar regions and more extensive in the bronchi and proximal bronchioles in the CCSP-rtTA compared to FoxA2-rtTA FoxA2Δ/Δ mice, consistent with the activity of the promoters. Goblet cell hyperplasia is more prominent in larger airways in the CCSP-rtTA deleted than the FoxA2rtTA-deleted mice, consistent with sites of gene targeting in the two models (FIG. 5).

Timed deletion of FoxA2 during lung morphogenesis. In order to determine the temporal requirements for FoxA2 during lung morphogenesis, dams or pups are treated with doxycycline at various time periods during development. Airspace enlargement, variable neutrophilic infiltration and goblet cell hyperplasia are detected in the FoxA2-rtTA, FoxA2Δ/Δ mice, while no pulmonary abnormalities are observed in the littermate controls. When the FoxA2-rtTA compound mice are treated with doxycycline postnatally, from PN1 to PN16, the extent of FoxA2 deletion is less and airspace enlargement is decreased compared to those maintained on doxycycline prenatally (FIG. 4). Airspace abnormalities are not observed in CCSP-rtTA-deleted mice (FIGS. 1 and 3).

Effects of FoxA2 deletion on epithelial cell gene expression and PECAM. Since FoxA2 influences the transcription of the Titf1, Sftpb, and Scgb1a1 genes in vitro (Bingle and Gitlin, 1993; Bingle et al., 1995; Bohinski et al., 1994; Ikeda et al., 1996), S1-nuclease protection assays are utilized to quantitate surfactant proteins (SP), SP-A, SP-B, FoxA2, and CCSP (Clara cell secretory protein) mRNAs at E17.5. When FoxA2-rtTA compound mice are maintained on doxycycline from E0 to E17.5-18, CCSP, SP-A, and SP-B mRNAs are significantly decreased (data not shown). Likewise, the content of SP-B in lung homogenates from surviving mice at PN16 is significantly decreased, 52.9±6% (mean±s.e.m., n=4) (supplemental FIG. 2). Since SP-B is critical for surfactant function, this decrease in SP-B may render the FoxA2-rtTA, FoxA2Δ/Δ mice more susceptible to respiratory dysfunction and death. Recent work from this laboratory demonstrated that reduction of SP-B to 20-30% of normal levels caused respiratory failure in adult mice (Melton et al., 2003). Immunohistochemical staining for CCSP is decreased in non-ciliated respiratory epithelial cells in the FoxA2Δ/Δ mice (supplemental FIG. 3). In contrast, FoxA2 mRNA, protein content, and immunostaining are unchanged, data not shown and (supplemental FIGS. 2 and 3). At E18.5, SP-A mRNA is reduced 12.7-fold and CCSP mRNA reduced 3-fold in the FoxA2-rtTA FoxA2Δ/Δ mice compared to controls. Immunostaining for TTF-1, mature SP-B, pro-FoxA2, T1α (a type I cell marker), FOXJ1 (a ciliated cell marker) and FOXA1 is not altered (FIG. 6 and supplemental FIG. 3). PECAM staining indicated normal distribution of pulmonary capillaries in the enlarged alveoli. Elastin staining is present in alveolar septa, however fewer septae are detected after FoxA2 deletion, indicating a primary abnormality in alveolarization-septation (FIG. 6). Elastin fibers are not fragmented or shortened, indicating that alterations in alveolar size in FoxA2Δ/Δ mice are not associated with elastin destruction. Extent and distribution of phosphohistone-3 staining, an indicator of cell proliferation, are unchanged in the FoxA2 deleted mice at E18.5 and PN2 (data not shown).

Pulmonary mechanics. Since most FoxA2-rtTA, FoxA2Δ/Δ mice died or are ill by maturity, lung mechanics are assessed on 7-week-old CCSP compound mice during forced oscillatory ventilation. Airway and tissue resistance and elastance are significantly increased and compliance decreased, suggesting abnormalities in both conducting airways and alveolar regions of the FoxA2Δ/Δ mice (FIG. 7).

Decreased FoxA2 staining in mouse models with goblet cell hyperplasia. Increased expression of IL-4 or IL-13, deletion of FoxA2 (Glasser et al., 2003; Jain-Vora et al., 1997; Kuperman et al., 2002; Rankin et al., 1996) and allergen challenge (Tomkinson et al., 2001) each cause pulmonary inflammation and goblet cell hyperplasia in vivo. We hypothesized that decreased expression of FoxA2 may contribute to the pathogenesis of goblet cell hyperplasia. FoxA2 staining is decreased or absent in goblet cells in the airways in each of these mouse models, supporting the concept that decreased FoxA2 is associated with or required for goblet cell hyperplasia (FIG. 6). Nuclear FoxA2 staining is decreased or absent in the surface cells with characteristics of goblet cells indicated by mucin, PAS and Alcian blue staining. FoxA2 staining is maintained in non-goblet bronchiolar epithelial cells and in basal cells that serve as precursors to goblet cells. Neither deletion of FoxA2 nor treatment with IL-13 altered phosphohistone-3 staining in the airways undergoing goblet cell hyperplasia, indicating that goblet cells are derived by differentiation of precursor cells (basal and Clara cells) rather than from proliferation (data not shown).

Effects of IL-4 on goblet cell hyperplasia and FoxA2 are Stat-6 dependent. Intratracheal administration of TH2 cytokines and IL-4 causes goblet cell hyperplasia in wild type but not in Stat-6−/− mice (Kuperman et al., 1998). In control mice, FoxA2 staining is decreased or absent in goblet cells after intratracheal administration of IL-4. In contrast, neither FoxA2 staining nor goblet cell hyperplasia are observed in the Stat-6−/− mice (FIG. 9).

Cytokine expression. To assess whether FoxA2 altered expression of cytokines known to cause lung inflammation or goblet cell hyperplasia, IL-4, IL-13, IL-5, IFN-γ, MIP-2, KC, and GM-CSF are measured by ELISA in lung homogenates from the FoxA2-rtTA compound mice at PN2 or PN9. While KC content is modestly but significantly increased from 40±3 pg/ml to 74±4 pg/ml mean±s.e.m., P=0.01, n=6 in the FoxA2 deleted mice, no statistically significant differences in the other cytokines are detected. Expression of mRNAs for cytokines known to be associated with lung inflammation and airway remodeling, including IL-4, 5, 6, 9, 10, 11, 13, 17, and TNF-α (Chen et al., 2003; Han et al., 1987; Matheson et al., 2002; Mishra et al., 2001; Rankin et al., 1996) are not altered in the FoxA2-rtTA, FoxA2Δ/Δ transgenic mice (data not shown).

FoxA2 inhibits transcription of the MUC5A/C gene in vitro. In order to assess whether FoxA2 directly regulated mucin expression in respiratory epithelial cells, a luciferase reporter construct containing 3.7 kb regulatory region of the mouse MUC5A/C gene is transfected with FoxA2 into H292 cells. FoxA2 significantly inhibited the activity of the MUC5A/C-luciferase construct in a dose dependent manner (FIG. 10), suggesting that FoxA2 inhibits gene expression associated with goblet cell phenotype.

Decreased FoxA2 associated with goblet cell hyperplasia in human lung disease. In order to determine the relationship between the loss of FoxA2 and goblet cell hyperplasia in humans, lung sections are obtained at autopsy or at lobectomy from 10 patients with chronic lung disease. Tissue is immunostained for FoxA2 and counterstained with Alcian blue. Five of the subjects are adults, four with cystic fibrosis and one with chronic pulmonary infection and bronchiectasis. Five subjects are infants dying in the first six months after birth with bronchopulmonary dysplasia. In all subjects, Alcian blue reactive, mucus producing cells lacked FoxA2 staining (FIG. 11A-C), while most cells lining normal airways stained for FoxA2. FoxA2 is readily detected in nuclei of adjacent, non-goblet, Alcian blue negative epithelial cells lining both conducting and terminal airways (FIG. 11D). Loss of FoxA2 is sufficient to cause goblet-cell hyperplasia in the absence of inflammatory stimuli.

Goblet cell hyperplasia and decreased FoxA2 staining. Goblet cell hyperplasia in the FoxA2Δ/Δ mice is associated with accumulation of both neutral and acidic mucins, increased MUC5A/C staining, and decreased CCSP staining at cellular sites in which FoxA2 is deleted in the conducting airways. In the mouse and human studies, goblet cells lacked FoxA2 staining while non-goblet columnar cells stained intensely, suggesting that effects of FoxA2 are cell autonomous rather than caused by secondary or reciprocal signaling among neighboring cells. Concentrations of lung cytokines known to be associated with goblet cell hyperplasia (IL-4, IL-13, and IL-5), and RNA levels of proinflammatory cytokines associated with goblet cell hyperplasia are not altered after deletion of FoxA2, suggesting that the loss of FoxA2 in airway epithelial cells directly influenced goblet cell hyperplasia in these models. The finding that FoxA2 inhibited transcriptional activity of the MUC5A/C gene in vitro, supports the concept that FoxA2 also directly inhibits mucin gene expression. Goblet cell hyperplasia following FoxA2 deletion is more extensive in proximal conducting airways in the CCSP-rtTA compound mice, consistent with the distinct sites of expression of the CCSP promoter used to express the rtTA and the sites of gene targeting in the two models. Goblet cell hyperplasia is seen in peripheral conducting airways in both models, again consistent with the sites of gene expression and recombination in the models (Perl et al., 2002a; Perl et al., 2002b; Stripp et al., 1992; Wert et al., 1993). In spite of extensive deletion of FoxA2 in subsets of cells in conducting airways and alveolar regions, not all FoxA2Δ/Δ cells become goblet cells, supporting the concept that additional factors influence mucus cell differentiation. Alternatively, it is unlikely that loss of FoxA2 influences goblet cell differentiation in distinct subsets of conducting airway cells.

Timing and sites of FoxA2 deletion influence airspace enlargement. Airspace enlargement is prominent in mice in which FoxA2 is deleted with the FoxA2-rtTA and is not seen in CCSP-rtTA transgenes. The timing and extent of recombination is distinct in the two models. Deletion of FoxA2 is extensive in the lung periphery in the FoxA2rtTA transgenic mice treated with doxycycline before birth, consistent with previous studies (Perl et al., 2002b). In the FoxA2-rtTA compound mice, deletion of FoxA2 occurs in lung progenitor cells and is extensive or complete as early as E6.5-8.5, prior to onset of branching morphogenesis. In contrast, the CCSP-rtTA transgene is not active until E14-15, targeting occurring primarily in the conducting airways rather than in the peripheral lung before birth (Perl et al., 2002a). Size of the lungs and the morphology of peripheral lung saccules is not altered prior to birth in the FoxA2Δ/Δ mice, whether deletion is induced with FoxA2- or CCSP-rtTA. Thus, FoxA2 is not required for prenatal lung morphogenesis, cell differentiation or perinatal survival, nor is it required for the expression of SP-B, a surfactant protein required for postnatal survival (Clark et al., 1995). Effects of FoxA2 deletion on peripheral lung morphogenesis are apparent as early as PN3, and extensive airspace enlargement is observed during alveolarization (postnatal days 10-20). Most FoxA2-rtTA, but not CCSP-rtTA, FoxA2Δ/Δ mice died or are ill after PN28. The finding that SP-B is significantly decreased provides a potential basis for increased susceptibility to lung dysfunction. Reduction of SP-B to 20-30% of normal causes respiratory failure in mice (Melton et al., 2003). The increased postnatal mortality seen in the FoxA2-rtTA, FoxA2Δ/Δ mice is likely related, at least in part, to the lack of SP-B. Most FoxA2Δ/Δ compound mice generated with FoxA2-rtTA are dead by one to two months of age. In contrast, survival of CCSP-rtTA, FoxA2Δ/Δ mice is unaltered during this time period. Elastin staining indicated deficient numbers of alveolar septae in the FoxA2, FoxA2Δ/Δ mice, demonstrating that FoxA2 plays a critical role in alveolarization. Abnormalities in peripheral airspaces generally occur before or in the absence of neutrophilic infiltrations and are not associated with fragmentation of elastin fibers.

We claim:

1. A method of treating pulmonary disease in a subject comprising the administration to a subject in need of such treatment a therapeutically effective amount of a formulation comprising a FoxA2 therapeutic.

2. The method of claim 1 wherein the FoxA2 therapeutic is an agent selected from the group consisting of an isolated FoxA2 protein, an isolated nucleic acid molecule encoding a FoxA2 protein, a FoxA2 receptor-specific antibody that stimulates the activity of the receptor, or pharmaceutically acceptable composition thereof.

3. The method of claim 2, wherein the FoxA2 therapeutic agent is a FoxA2 receptor-specific antibody that stimulates the activity of the receptor.

4. The method of claim 2, wherein the FoxA2 therapeutic agent is an isolated FoxA2 protein or proFoxA2 protein.

5. The method of claim 2, wherein the FoxA2 therapeutic agent is an isolated nucleic acid molecule encoding a FoxA2 protein or proFoxA2 protein, wherein the nucleic acid molecule is operatively linked to a transcription control sequence.

6. The method of claim 5, wherein the nucleic acid molecule is expressed in the subject's airway cells.

7. The method of claim 6, wherein the nucleic acid that encodes a FoxA2 polypeptide, fragment, homolog or variant with substantial homology, supplying FoxA2 function.

8. The method of claim 7, wherein the nucleic acid molecule becomes integrated to the chromosomal DNA making up the genome of the subject's airway cells.

9. The method of claim 7, wherein the nucleic acid molecule is expressed by the subject's airway cells from an extrachromosomal location.

10. The method of claim 7, wherein the nucleic acid molecule comprises at least 50 nucleotides.

11. The method of claim 7, wherein the nucleic acid molecule comprises at least 200 nucleotides.

12. The method of claim 7, wherein the airway cells are selected from the group consisting of smooth muscle and epithelial cells.

13. The method of claim 7, wherein the isolated nucleic acid molecule is administered to the mammal complexed with a liposome delivery vehicle.

14. The method of claim 7, wherein the isolated nucleic acid molecule is administered to the mammal in a viral vector delivery vehicle.

15. The method of claim 14, wherein the viral vector delivery vehicle is from adenovirus.

16. The method of claim 7, wherein the isolated nucleic acid molecule, when administered to the lungs of the mammal, is expressed in cells of the mammal.

17. The method of claim 2, wherein the disease is a chronic obstructive pulmonary disease of the airways associated with eosinophilic inflammation.

18. The method of claim 2, wherein the disease is selected from the group consisting of airway obstruction, allergies, asthma, acute inflammatory lung disease, chronic inflammatory lung disease, chronic obstructive pulmonary dysplasia, emphysema, pulmonary emphysema, chronic obstructive emphysema, adult respiratory distress syndrome, bronchitis, chronic bronchitis, chronic asthmatic bronchitis, chronic obstructive bronchitis, and interstitial lung diseases.

19. The method of claim 2, wherein the FoxA2 therapeutic agent decreases lung inflammation in the mammal.

20. The method of claim 2, wherein the FoxA2 therapeutic agent is administered in an amount between about 0.1 micrograms/kilogram and about 10 milligram/kilogram body weight of a mammal.

21. The method of claim 2, wherein the FoxA2 therapeutic agent is administered in a pharmaceutically acceptable excipient.

22. The method of claim 2, wherein the mammal is a human.

23. The method of claim 1, wherein the FoxA2 therapeutic agent is administered by at least one route selected from the group consisting of nasal and inhaled routes.

24. The method of claim 2, wherein the pulmonary disease is selected from the group consisting of asthma, allergic bronchopulmonary aspergillosis, hypersensitivity pneumonia, eosinphilic pneumonia, allergic bronchitis bronchiectasis, hypersensitivity pneumotitis, occupational asthma, reactive airway disease syndrome, hypereosinophilic syndrome, rhinitis, sinusitis, and parasitic lung disease.

25. A method for prescribing treatment for airway hyperresponsiveness and/or airflow limitation associated with a respiratory disease involving an inflammatory response in a mammal, comprising: a. administering to the lungs of a mammal a FoxA2 therapeutic agent selected from the group consisting of: a FoxA2 receptor-specific antibody that stimulates the activity of the receptor an isolated FoxA2 protein or proFoxA2 protein; and an isolated nucleic acid molecule encoding a FoxA2 protein or proFoxA2 protein, wherein the nucleic acid molecule is operatively linked to a transcription control sequence; b. measuring a change in lung function in response to a provoking agent in the mammal to determine if the FoxA2 therapeutic agent modulates airway hyperresponsiveness; and c. prescribing a pharmacological therapy comprising administration of FoxA2 therapeutic agent to the mammal effective to reduce inflammation based upon the changes in lung function.

26. A method of prophylactically treating a subject at risk of developing an airway hyperresponsiveness and/or airflow limitation associated with a respiratory disease involving an inflammatory response, the method comprising administering to the subject a therapeutically effective amount of a formulation comprising a FoxA2 therapeutic demonstrated to inhibit onset of the respiratory disease.

27. A method of improving gas exchange in the lungs of a subject comprising identifying a subject for whom an improvement in gas exchange within the lungs would be beneficial; providing a therapeutically effective amount of a formulation comprising a FoxA2 therapeutic demonstrated to impove gas exchange within the lungs to the subject for inhalation, and before, during, or immediately after the FoxA2 therapeutic is inhaled by the subject, introducing into the subject a therapeutically-effective amount an anti-inflammatory agent.

28. The method of claim 27, wherein the anti-inflammatory agent is selected from the group consisting of anti-IgE, immunomodulating drugs, leukotriene synthesis inhibitors, leukotriene receptor antagonists, glucocorticosteroid, steroid chemical derivatives, anti-cyclooxygenase agents, beta-adrenergic agonists, methylxanthines, cromones, anti-CD4 reagents, anti-1L-5 reagents, surfactants, cytoxin, and heparin.

29. The method of claim 27, wherein the anti-inflammatory agent is a glucocorticoid selected from the group consisting of budesonide, rofleponide, beclomethasone dipropionate, beclomethasone monopropionate, ciclesonide, tipredane, flunisolide, triamcinolone acetonide, and fluticasone propionate, wherein said glucocorticoid substance is released in the lower part of the small intestine or the upper part of the large intestine, and wherein the glucocorticoid substance is administered in an amount that is therapeutically effective for the treatment of airway inflammation in the patient.

30. The method of claim 27, wherein the subject is hypoxic.

31. The method of claim 27, wherein the subject is a human suffering from a lung injury.

32. A formulation for protecting a mammal from airway hyperresponsiveness, airflow limitation and/or airway fibrosis associated with a respiratory disease involving inflammation, comprising an anti-inflammatory agent effective for reducing eosinophilic inflammation and a FoxA2 therapeutic agent selected from the group consisting of: a FoxA2 receptor-specific antibody that stimulates the activity of the receptor; an isolated FoxA2 protein or proFoxA2 protein; and an isolated nucleic acid molecule encoding a FoxA2 protein or proFoxA2 protein, wherein the nucleic acid molecule is operatively linked to a transcription control sequence.

33. The formulation of claim 32, wherein the formulation comprises a pharmaceutically acceptable excipient.

34. The formulation of claim 32, wherein the formulation comprises a controlled release vehicle selected from the group consisting of biocompatible polymers, other polymeric matrices, capsules, microcapsules, microparticles, bolus preparations, osmotic pumps, diffusion devices, liposomes, lipospheres, viral vectors and transdermal delivery systems.

35. The formulation of claim 32, wherein the FoxA2 therapeutic agent is an isolated FoxA2 protein or proFoxA2 protein.

36. The formulation of claim 32, wherein the FoxA2 therapeutic agent is an isolated nucleic acid molecule encoding a FoxA2 protein or proFoxA2 protein, wherein the nucleic acid molecule is operatively linked to a transcription control sequence.

37. The formulation of claim 36, wherein the isolated nucleic acid molecule is complexed with a liposome delivery vehicle.

38. The formulation of claim 36, wherein the isolated nucleic acid molecule in a viral vector delivery vehicle.

39. The formulation of claim 38, wherein the viral vector delivery vehicle is from adenovirus.

40. The formulation of claim 32, wherein the FoxA2 therapeutic agent is a FoxA2 receptor-specific antibody that stimulates the activity of the receptor.

41. The formulation of claim 32, wherein the FoxA2 therapeutic agent is selected from the group consisting of: an isolated FoxA2 protein or proFoxA2 protein and an isolated nucleic acid molecule encoding a FoxA2 protein or proFoxA2 protein, wherein the nucleic acid molecule is operatively linked to a transcription control sequence.

42. The formulation of claim 32, wherein the anti-inflammatory agent is selected from the group consisting of anti-IgE, immunomodulating drugs, leukotriene synthesis inhibitors, leukotriene receptor antagonists, glucocorticosteroids, steroid chemical derivatives, anti-cyclooxygenase agents, beta-adrenergic agonists, methylxanthines, cromones, anti-CD4 reagents, anti-IL-5 reagents, surfactants, cytoxin, and heparin.

43. The composition according to claim 32 further comprising a phospholipid selected from the group consisting of phosphatidylcholine, phosphatidylethanolamine, hydroxylated lecithin, distearoylphosphatidylcholine, phosphatidylserine, phosphatidylglycerol, phosphatidic acid, phosphatidylinositol, sphingomyelin, dimyristoylphosphatidylcholine, dimyristoylphosphatidylglycerol, and mixtures thereof in an amount ranging from about 1% to about 20% by weight of said composition.


SEQUENCE LISTING (7 sequences)

SEQ ID NO: 1 - DNA, 2242 bp, homo sapiens
  CDS (204)...(1577)
  misc_feature (678)...(965): Fork Head Domain
cccgcccacttccaactaccgcctccggcctgcccagggagagagagggagtggagcccagggagagggagcgcgagagagggagggaggaggggacggtgctttggctgacttttttttaaaagagggtgggggtggggggtgattgctggtcgtttgttgtggctgttaaattttaaactgccatgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggtttt

SEQ ID NO: 2 - protein, 457 aa, homo sapiens
  DOMAIN (159)...(254): Fork Head Domain
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 3 - DNA, 2230 bp, homo sapiens
  CDS (192)...(1565)
  misc_feature (666)...(953): Fork Head Domain
tcggccgctgctagaggggctgcttgcgccaggcgccggccgccccactgcgggtccctggcggccggtgtctgaggagtcggagagccgaggcggccagaccgtgcgccccgcgcttctcccgaggccgttccgggtctgaactgtaacagggaggggcctcgcaggagcagcagcgggcgagttaaagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggtttt

SEQ ID NO: 4 - DNA, 1001 bp, Mus musculus
  promoter (1)...(1001): upstream promoter for FoxA1 from -1000 to 0
ccggcagagaggcaggcttgactaggatccgcctgccttatcacacaaacatctgcgcgcagtttcttcctctttctgatcgctgcagttgacaaggggagtggagtggactgaggcggaaaaaaaaagttatgtcataaaaatataaaacggtgctgtgactcacctgctcttagccgcaggtacgagtttcgtggcagcgcctccgttctgctcgccaggagcccaggtcggcctaacgtcactttgctcggctgactcagatgactgggtttctagtgacgaggcaaaagtggggcttttcccctctccaagccccgaaggccgggaagtaggggtagtcttccagggttgaactttgggtcaagaatcaggatgaggggagcaaaggagccaagtggaccaagagcggagtcgtagtaaggtccagggggtggtgagcacctctaggagggaactcggggtcattcagcgagcacgatccttgcgctgtagctctgatgccaccacccaggtccttctcccgcagcacagctctttgggtccagagccctggcctgtcctccaaggcaccgcctatttttccttttcttcttttttttccttttcttttctttctctttttttccttttccaaagggggtcacacacacacccgccctattttcctctttccctagccgcgaggcttaaaccaattatactgctttgtaaacaaagtgagggccaggtttgggggaggggatggcgggaggggcgcggggggcgcgcggcgctggcgcggcggggcgggaggcgcggcggctggactggcgggcggccgcctcacaggtgcacctcgggctttgtaggtgcgagcgtctttgtgcggcggacaaatggggagaggacgaggaggtgggcactccggcgacgtaagatccacatcagctcaactgcactcgcttcgcacaggccgcccgctcacttcccgcggaggcgctgccgggcgccgcgctccg

SEQ ID NO: 5 - DNA, 1001 bp, Mus musculus
  promoter (1)...(1001): upstream promoter for FoxA2 from -1000 to 0
atctgatctctctgggttccctgtgttcactaggtgaacacgtcagattcaccttcagcgggaaaacaggcgttgcctttgagggactcgtgctctgcattgcttcagatgacacattaataccttatctgtccatactatgtgcgtttcagcagcaaagggcactgatgttgctgcgtcttcagtccaaaaggatgctgacctctttctttaccctccaatctggcttccattctcattcaggcttccatgctcaaagctcaccccagtacctgccacctctatgtactcttcttctaccacacgaaattgtggacacctggatttgggccaactatttcctattttctccatctcctagagagaagagtttgggactgtatccctacatcctagtcacattttcagaaccaagaaagactgtccacataaaaacgccggcaagaccctccactccaaaatctaggcttaccttagtctcggtcttggtagctaacaatataaatgacatactctgttgttttcatgtttgttttgtttggggagacaaggtttctctctgtagcccggatgtcttgaaactcactctatagactaggtcagcctagacttctctgagatcctcctgcctctgcctcaaaggcagaagacaacacttcaaatgacactttttaacacctagaagctaaagagaaaagaaactgaaattttcaatttttataagacagtcctggtctctgcaggcagagaacacagatcctcctgaagtcatcccacaaggcccattattgattttttttctcctgccctacccccacctactgccctgtttgttttagttacgaaatgctttgggcaccttggatttaactgaaaagtaaccttgaaacaccgaggccctcatgccagaggcaaatcgctgcctcccgggtattggctgcagctaaacgggtctctccaggccgactgaggtgggtagccagaaagaggactgaggtaac

SEQ ID NO: 6 - DNA, 1002 bp, homo sapiens
  promoter (1)...(1001): upstream promoter for FoxA1 from -1000 to 0
tgttatgggagatgtcccctccccctgaccttttcctatcagaaaccagacggtcgtctccagctcactccctgaagcggctgaaggggggcgggaggaggagcccggcagggggaggacggcccgacttgggtccgcctgccctatcacacagacatctgctctcagtttcttcctctttgtggtcgctacagatgacaaggggagaggagcggaccgaggcggaacaaaaaaaaaaagttatgtcataaagatataaaccggtgctgtgactcacctgctcttagccgcaggtacgagtttcgtggcagcgcctccgctctgctagcccggggcccgggtcggcctaacgtcacttggctcggctgactcagatgacccggtttctagtgacgaagcaaaagtggggctttttcccttttctagtctgaagctctcaggaggaaagaaaaaaaaaagtctgtattctccagtctcccggggttgaactttgggtcaggaatcaggagggaggaagcaaagagcaggaggggcgcgagactgggccgcagcccgcgctcggaaggtggcgtgtacctctggggagggaccccggggtcagttatcccacaaggtctctaagcccagtctccgacgccaccgcccccggccttctccggctgcgaggttccgatccagagccctagcccgcccgcccgcctggccccgcctattttttctttttccaaagggggttacaccccctccctccccatttccctctttccctagcctccgggcctaaaccaattacactgctttgcaaacaaagtgagggccccgcttgggggaggggtcgcgggggaggggtcgcgcgcagcggtgaagcggcggggcgggaggcgcggcggccggactggcgggcggccgccttgcaggtgcacctcgggctttgtaggtgcgagcgtctttgtgcggcggacaaatggggagaggacgaggaggtgggcactcaagcgacgt

SEQ ID NO: 7 - DNA, 1001 bp, homo sapiens
  promoter (1)...(1001): upstream promoter for FoxA2 from -1000 to 0
cggccgctgctagaggggctgcttgcgccaggcgccggccgccccactgcgggtccctggcggccggtgtctgaggagtcggagagccgaggcggccagaccgtgcgccccgcgcttctcccgaggccgttccgggtctgaactgtaacagggaggggcctcgcaggagcagcagcgggcgagttaaaggtgtgtacgtagtttttctaaataggacagcgctttgcaaattggctgtcatggacttgctgttttgacagagaatgagcactgagagcgaagaaacccggcataaataaatccggctttcatagggatctgccgggcactgaggctggacgcgccctccgacaggcgcaccgggcgcccggcgcgatgagacggggcgggggctggaggccggagccgccccagagggtggtttcctcccaggtccctccttcctcaggggactcgctgtggacccgggaggaaagggggctcgaccagggatcccgcgtgcccgatcgcccgcccagcccgctcccctggcccgcccaggagtgcggggcgccgcggcggtggggcggacggcaggcgctgaggggcaggtgcggagcgccggctgcggctacctctcacctaggtgtgagctgattattcaaatgggctgccccgggtctggggattggaggcccgcgccagcgcgccgcgctatatcaccagcctcccacgtcactgcggcacttgtccgctccgcggtccacacctcctcctcctcctcctcctcctcctcctcctcctcctcctccaccaccaccaccgcctcctcctcctccttcctcctcctcctccccacccccccactagcccccgcgccgcgcgcttcccgccgcctcggggcagctcggcactgccaacctcagccgccacccggcagcccgtgagcgcggcggcggcgggtgctccctaccgcggggcgctggcctgggcctcctccgctcctcgcccccccccacctc